(19) United States
(12) Patent Application Publication
(10) Pub. No.: US 2004/0092561 A1
(43) Pub. Date: May 13, 2004

(54) Azolidinone-vinyl fused -benzene derivatives

(76) Inventors: Thomas Ruckle, Plan-les-Ouates (CH); Tania Valloton, Corsier (CH); Pascale Gaillard, Collonge-sous-Saleve (FR); Dennis Church, Commugny (CH); Xuliang Jiang, Braintree, MA (US)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/289,998

(22) Filed: Nov. 7, 2002

(51) Int. Cl. A61K 31/426; A61K 31/421; A61K 31/4164

(52) U.S. Cl. 514/369; 514/376; 514/389

(57) ABSTRACT

The present invention is related to azolidinedione-vinyl fused-benzene derivatives of formula (I) for the treatment and/or prophylaxis of autoimmune disorders and/or inflammatory diseases, cardiovascular diseases, neurodegenerative diseases, bacterial or viral infections, kidney diseases, platelet aggregation, cancer, transplantation, graft rejection or lung injuries.

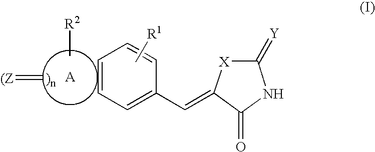

wherein A, X, Y, Z, R¹, R² and n are as described in the description.

### FIELD OF THE INVENTION

This present invention is related to the use of azolidinone-vinyl fused-benzene derivatives of formula (I) for the treatment and/or prophylaxis of autoimmune disorders and/or inflammatory diseases, cardiovascular diseases, neurodegenerative diseases, bacterial or viral infections, kidney diseases, platelet aggregation, cancer, transplantation, graft rejection or lung injuries. Specifically, the present invention is related to substituted azolidinone-vinyl fused-benzene derivatives for the modulation, notably the inhibition of the activity or function of the phospho-inositide-3′OH kinase family, PI3K, particularly of the PI3Kγ.

### BACKGROUND OF THE INVENTION

Cellular plasma membranes can be viewed as a large store of second messenger that can be enlisted in a variety of signal transduction pathways. As regards function and regulation of effector enzymes in phospholipid signalling pathways, these enzymes generate second messengers from the membrane phospholipid pool (class I PI3 kinases (e.g. PI3Kgamma)) are dual-specific kinase enzymes, means they display both: lipid kinase (phosphorylation of phospho-inositides) as well as protein kinase activity, shown to be capable of phosphorylation of other protein as substrates, including auto-phosphorylation as intra-molecular regulatory mechanism. These enzymes of phospholipid signalling are activated in response to a variety of extra-cellular signals such as growth factors, mitogens, integrins (cell-cell interactions) hormones, cytokines, viruses and neurotransmitters such as described in Scheme 1 hereinafter and also by intra-cellular cross regulation by other signaling molecules (cross-talk, where the original signal can activate some parallel pathways that in a second step transmitt signals to PI3Ks by intra-cellular signaling events), such as small GTPases, kinases or phosphatases for example.

The inositol phospholipids (phosphoinositides) intracellular signalling pathway begins with binding of a signalling molecule (extracellular ligands, stimuli, receptor dimerization, transactivation by heterologous receptor (e.g. receptor tyrosine kinase)) to a G-protein linked transmembrane receptor integrated into the plasma membrane. PI3K converts the membrane phospholipid PIP(4,5)2 into PIP(3,4,5)3 which in turn can be further converted into another 3′ phosphorylated form of phosphoinositides by 5′-specific phospho-inositide phosphatases, thus PI3K enzymatic activity results either directly or indirectly in the generation of two 3′-phosphoinositide subtypes that function as 2^{nd} messengers in intra-cellular signal transduction (Trends Biochem Sci. 22(7) p.267-72 (1997) by Vanhaesebroeck B et al., Chem Rev. 101(8) p.2365-80 (2001) by Leslie N. R et al (2001); Annu Rev Cell Dev Biol. 17 p.615-75 (2001) by Katso R. et al. and Cell Mol Life Sci. 59(5) p.761-79 (2002) by Toker a. et al.). Multiple PI3K isoforms categorized by their catalytic subunits, their regulation by corresponding regulatory subunits, expression patterns and signaling-specific functions (p110α, β, δ, and γ) perform this enzymatic reaction (Exp Cell Res. 25(1) p.239-54 (1999) by Vanhaesebroeck B. and Annu Rev Cell Dev Biol. 17 p.615-75 (2001) by Katso R. et al).

The evolutionary conserved isoforms p110 α and β are ubiquitiously expressed, while δ and γ are more specifically expressed in the haematopoetic cell system, smooth muscle cells, myocytes and endothelial cells (Trends Biochem Sci. 22(7) p.267-72 (1997) by Vanhaesebroeck B et al.). Their expression might also be regulated in an inducible manner depending on the cellular-, tissue type and stimuli as well as disease context. To date, eight mammalian PI3Ks have been identified, divided into three main classes (I, II, and III) on the basis of sequence homology, structure, binding partners, mode of activation, and substrate preference in vitro. Class I PI3Ks can phosphorylate phosphatidylinositol (PI), phosphatidylinositol-4-phosphate, and phosphatidylinositol-4,5-biphosphate (PIP2) to produce phosphatidylinositol-3-phosphate (PIP), phosphatidylinositol-3,4-biphosphate, and phosphatidylinositol-3,4,5-triphosphate, respectively. Class II PI3Ks phosphorylate PI and phosphatidylinositol-4-phosphate. Class III PI3Ks can only phosphorylate PI (Trends Biochem Sci. 22(7) p.267-72 (1997) by Vanhaesebroeck B et al, Exp Cell Res. 25(1) p.239-54 (1999) by Vanhaesebroeck B. and Chem Rev. 101(8) p.2365-80 (2001) by Leslie N. R et al (2001)) G-protein coupled receptors mediated phosphoinositide 3′OH-kinase activation via small GTPases such as Goy and Ras, and consequently PI3K signaling plays a central role in establishing and coordinating cell polarity and dynamic organization of the cytoskeleton—which together provides the driving force of cells to move.

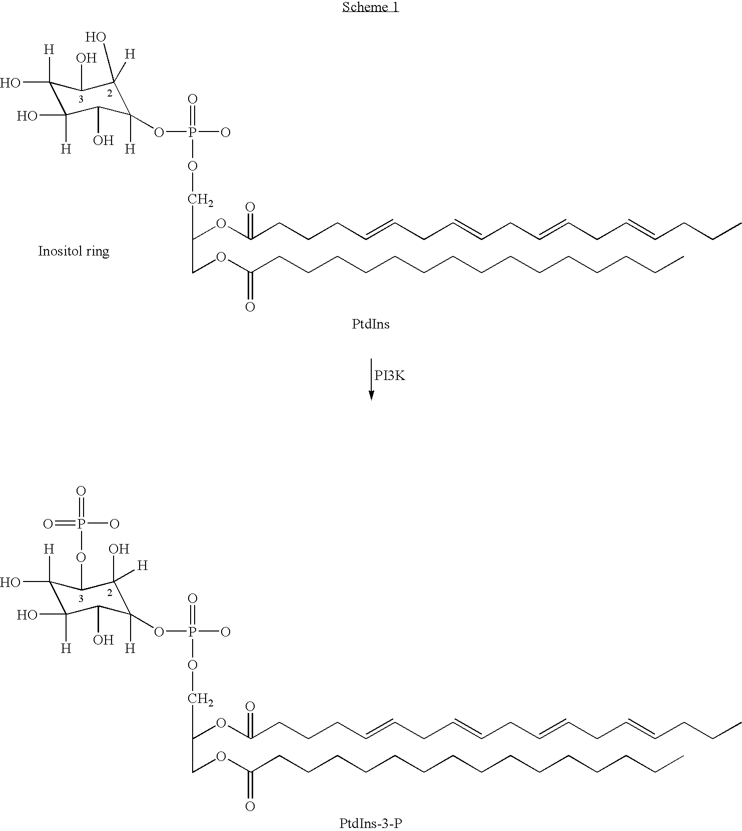

As above illustrated in Scheme 1, Phosphoinositide 3-kinase (PI3K) is involved in the phosphorylation of Phosphatidylinositol (PtdIns) on the third carbon of the inositol ring. The phosphorylation of PtdIns to 3,4,5-triphosphate (PtdIns(3,4,5)P₃), PtdIns(3,4)P₂ and PtdIns(3)P act as second messengers for a variety of signal transduction pathways, including those essential to cell proliferation, cell differentiation, cell growth, cell size, cell survival, apoptosis, adhesion, cell motility, cell migration, chemotaxis, invasion, cytoskeletal rearrangement, cell shape changes, vesicle trafficking and metabolic pathway (Annu Rev Cell Dev Biol. 17 p.615-75 (2001) by Katso et al. and Mol Med Today 6(9) p.347-57 (2000) by Stein R. C). Chemotaxis—the directed movement of cells toward a concentration gradient of chemical attractants, also called chemokines is involved in many important diseases such as inflammation/auto-immunity, neurodegeneration, angiogenesis, invasion/metastisis and wound healing (Immunol Today 21(6) p.260-4 (2000) by Wyman N P et al.; Science 287(5455) p.1049-53 (2000) by Hirsch et al.; FASEB J 15(11) p.2019-21 (2001) by Hirsch et al. and Nat Immunol. 2(2) p.108-15 (2001) by Gerard C. et al.).

Recent advances using genetic approaches and pharmacological tools have provided insights into signaling and molecular pathways that mediate chemotaxis in response to chemoattractant activated G-protein coupled receptors PI3-Kinase, responsible for generating these phosphorylated signalling products, was originally identified as an activity associated with viral oncoproteins and growth factor receptor tyrosine kinases that phosphorylates phosphatidylinositol (PI) and its phosphorylated derivatives at the 3′-hydroxyl of the inositol ring (Panayotou et al., Trends Cell Biol. 2 p.358-60 (1992)). However, more recent biochemical studies revealed that, class I PI3 kinases (e.g. class IB isoform PI3Kγ) are dual-specific kinase enzymes, means they display both: lipid kinase (phosphorylation of phospho-inositides) as well as protein kinase activity, shown to be capable of phosphorylation of other protein as substrates, including auto-phosphorylation as intra-molecular regulatory mechanism.

So, PI3-kinase activation, therefore, is believed to be involved in a range of cellular responses including cell growth, differentiation, and apoptosis (Parker et al., Current Biology, 5 p.577-99 (1995), Yao et al., Science, 267 p.2003-05 (1995)). PI3-kinase appears to be involved in a number of aspects of leukocyte activation. A p85-associated PI3-kinase activity has been shown to physically associate with the cytoplasmic domain of CD28, which is an important costimulatory molecule for the activation of T-cells in response to antigen (Pages et al., Nature, 369 p,327-29 (1994); Rudd, Immunity 4 p.527-34 (1996)). Activation of T cells through CD28 lowers the treshold for activation by antogen and increases the magnitude and duration of the proliferative response. These effects are linked to increases in the transcription of a number of genes including interleukin-2 (IL2), an important T cell growth factor (Fraser et al., Science, 251 p.313-16 (1991)). Mutation of CD28 such that it can longer interact with PI3-kinase leads to a failure to initiate IL2 production, suggesting a critical role for PI3-kinase in T cell activation. PI3Kγ has been identified as a mediator of G beta-gamma-dependent regulation of JNK activity, and G beta-gamma are subunits of heterotrimeric G proteins (J. Biol. Chem. 273(5) p.2505-8 (1998). Cellular processes in which PI3Ks play an essential role include suppression of apoptosis, reorganization of the actin skeleton, cardiac myocyte growth, glycogen synthase stimulation by insulin, TNFα-mediated neutrophil priming and superoxide generation, and leukocyte migration and adhesion to endothelial cells.

Recently, (Immunity 16(3) p.441-51 (2002)) it has been described that PI3Kγ relays inflammatory signals through various G(i)-coupled receptors and its central to mast cell function, stimuli in context of leukocytes, immunology includes cytokines, chemokines, adenosines, antibodies, integrins, aggregation factors, growth factors, viruses or hormones for example (J.Cell. Sci. 114(Pt 16) p.2903-10 (2001) by Lawlor M A et al., Immunity 16(3) p.441-51 (2002) by Laffargue M. et al. and Curr. Opinion Cell Biol. 14(2) p.203-13 (2002) by Stephens L. et al.).

Specific inhibitors against individual members of a family of enzymes provide invaluable tools for deciphering functions of each enzyme. Two compounds, LY294002 and wortmannin (cf.hereinafter), have been widely used as PI3-kinase inhibitors. These compounds are non-specific PI3K inhibitors, as they do not distinguish among the four members of Class I PI3-kinases. For example, the IC₅₀ values of wortmannin against each of the various Class I PI3-kinases are in the range of 1-10 nM. Similarly, the IC₅₀ values for LY294002 against each of these PI3-kinases is about 15-20 μM (Fruman et al., An. Rev. Biochem., 67 p.481-507 (1998)), also 5-10 microM on CK2 protein kinase and some inhibitory activity on phospholipases. Wortmannin is a fungal metabolite which irreversibly inhibits PI3K activity by binding covalently to the catalytic domain of this enzyme. Inhibition of PI3K activity by wortmannin eliminates the subsequent cellular response to the extracellular factor. For example, neutrophils respond to the chemokine fMet-Leu-Phe (fMLP) by stimulating PI3K and synthesizing PtdIns (3, 4, 5)P₃. This synthesis correlates with activation of the respirators burst involved in neutrophil destruction of invading microorganisms. Treatment of neutrophils with wortmannin prevents the fMLP-induced respiratory burst response (Thelen et al. PNAS 91 p.4960-64 (1994)). Indeed, these experiments with wortmannin, as well as other experimental evidence, shows that PI3K activity in cells of hematopoietic lineage, particularly neutrophils, monocytes, and other types of leukocytes, is involved in many of the non-memory immune response associated with acute and chronic inflammation.

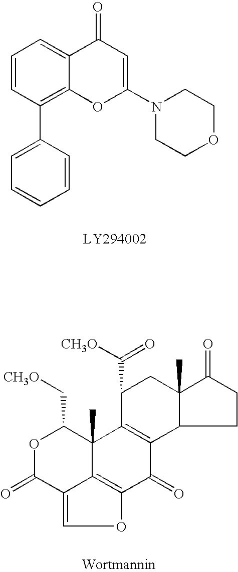

Based on studies using wortmannin, there is evidence that PI3-kinase function also is required for some aspects of leukocyte signaling through G-protein coupled receptors (Thelen et al., Proc. Natl. Acad. Sci. USA, 91 p.4960-64 (1994)). Morever, it has been shown that wortmannin and LY294002 block neutrophil migration and superoxide release. However, in as much as these compounds do not distinguish among the various isoforms of PI3K, it remains unclear which particular PI3K isoform or isoforms are involved in these phenomena.

### SUMMARY OF THE INVENTION

The present invention relates to the use of azolidinone-vinyl fused-benzene derivatives of formula (I)

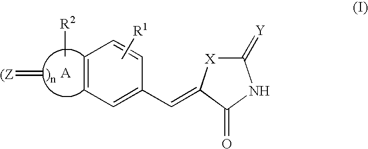

wherein A, X, Y, Z, n, R¹ and R² are described in details in the description hereinafter, as well as pharmaceutically acceptable salts thereof, for the preparation of pharmaceutical compositions for the treatment and/or prophylaxis of autoimmune disorders and/or inflammatory diseases, cardiovascular diseases, neurodegenerative diseases, bacterial or viral infections, kidney diseases, platelet aggregation, cancer, transplantation, graft rejection or lung injuries. Compounds of this invention are inhibitors of Phosphato-inositides 3-kinases (PI3Ks), particularly of Phosphatoinositides 3-kinases gamma (PI3Kγ).

### DESCRIPTION OF THE INVENTION

It has now been found that compounds of the present invention are modulators of the Phosphatoinositides 3-kinases (PI3Ks), particularly of Phosphatoinositides 3-kinase γ (PI3Kγ). When the phosphatoinositides 3-kinase (PI3K) enzyme is inhibited by the compounds of the present invention, PI3K is unable to exert its enzymatic, biological and/or pharmacological effects. The compounds of the present invention are therefore useful in the treatment and prevention of autoimmune disorders and/or inflammatory diseases, cardiovascular diseases, neurodegenerative diseases, bacterial or viral infections, kidney diseases, platelet aggregation, cancer, transplantation, graft rejection or lung injuries.

The following paragraphs provide definitions of the various chemical moieties that make up the compounds according to the invention and are intended to apply uniformly through-out the specification and claims unless an otherwise expressly set out definition provides a broader definition.

“C₁-C₆-alkyl” refers to monovalent alkyl groups having 1 to 6 carbon atoms. This term is exemplified by groups such as methyl, ethyl, n-propyl, isopropyl, n-butyl, isobutyl, tert-butyl, n-hexyl and the like.

“Aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenantrenyl and the like.

“C₁-C₆-alkyl aryl” refers to C₁-C₆-alkyl groups having an aryl substituent, including benzyl, phenethyl and the like.

“Heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl,1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

“C₁-C₆-alkyl heteroaryl” refers to C₁-C₆-alkyl groups having a heteroaryl substituent, including 2-furylmethyl, 2-thienylmethyl, 2-(1H-indol-3-yl)ethyl and the like.

“C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl (allyl, —CH₂CH═CH₂) and the like.

“C₂-C₆-alkenyl aryl” refers to C₂-C₆-alkenyl groups having an aryl substituent, including 2-phenylvinyl and the like.

“C₂-C₆-alkenyl heteroaryl” refers to C₂-C₆-alkenyl groups having a heteroaryl substituent, including 2-(3-pyridinyl)vinyl and the like.

“C₂-C₆-alkynyl” refers to alkynyl groups preferably having from 2 to 6 carbon atoms and having at least 1-2 sites of alkynyl unsaturation, preferred alkynyl groups include ethynyl (—C≡CH), propargyl (—CH₂C≡CH), and the like.

“C₂-C₆-alkynyl aryl” refers to C₂-C₆-alkynyl groups having an aryl substituent, including phenylethynyl and the like.

“C₂-C₆-alkynyl heteroaryl” refers to C₂-C₆-alkynyl groups having a heteroaryl substituent, including 2-thienylethynyl and the like.

“C₃-C₈-cycloalkyl” refers to a saturated carbocyclic group of from 3 to 8 carbon atoms having a single ring (e.g., cyclohexyl) or multiple condensed rings (e.g., norbornyl). Preferred cycloalkyl include cyclopentyl, cyclohexyl, norbornyl and the like.

“Heterocycloalkyl” refers to a C₃-C₈-cycloalkyl group according to the definition above, in which up to 3 carbon atoms are replaced by heteroatoms chosen from the group consisting of O, S, NR, R being defined as hydrogen or methyl. Preferred heterocycloalkyl include pyrrolidine, piperidine, piperazine, 1-methylpiperazine, morpholine, and the like.

“C₁-C₆-alkyl cycloalkyl” refers to C₁-C₆-alkyl groups having a cycloalkyl substituent, including cyclohexylmethyl, cyclopentylpropyl, and the like.

“C₁-C₆-alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like.

“Carboxy” refers to the group —C(O)OH.

“C₁-C₆-alkyl carboxy” refers to C₁-C₆-alkyl groups having an carboxy substituent, including 2-carboxyethyl and the like.

“Acyl” refers to the group —C(O)R where R includes “C₁-C₆-alkyl”, “aryl”, “heteroaryl”, “C₁-C₆-alkyl aryl” or “C₁-C₆-alkyl heteroaryl”.

“C₁-C₆-alkyl acyl” refers to C₁-C₆-alkyl groups having an acyl substituent, including 2-acetylethyl and the like.

“Aryl acyl” refers to aryl groups having an acyl substituent, including 2-acetylphenyl and the like.

“Heteroaryl acyl” refers to hetereoaryl groups having an acyl substituent, including 2-acetylpyridyl and the like.

“C₃-C₈-(hetero)cycloalkyl acyl” refers to 3 to 8 memebered cycloalkyl or heterocycloalkyl groups having an acyl substituent.

“Acyloxy” refers to the group —OC(O)R where R includes H, “C₁-C₆-alkyl”, “C₂-C₆-alkenyl”, “C₂-C₆-alkynyl”, “C₃-C₈-cycloalkyl”, heterocycloalkyl“heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl“, ”C₁-C₆-alkyl cycloalkyl”, “C₁-C₆-alkyl heterocycloalkyl”.

“C₁-C₆-alkyl acyloxy” refers to C₁-C₆-alkyl groups having an acyloxy substituent, including 2-(acetyloxy)ethyl and the like.

“Alkoxy” refers to the group —O—R where R includes “C₁-C₆-alkyl” or “aryl” or “heteroaryl” or “C₁-C₆-alkyl aryl” or “C₁-C₆-alkyl heteroaryl”. Preferred alkoxy groups include by way of example, methoxy, ethoxy, phenoxy and the like.

“C₁-C₆-alkyl alkoxy” refers to C₁-C₆-alkyl groups having an alkoxy substituent, including 2-ethoxyethyl and the like.

“Alkoxycarbonyl” refers to the group —C(O)OR where R includes H, “C₁-C₆-alkyl” or “aryl” or “heteroaryl” or “C₁-C₆-alkyl aryl” or “C₁-C₆-alkyl heteroaryl”.

“C₁-C₆-alkyl alkoxycarbonyl” refers to C₁-C₅-alkyl groups having an alkoxycarbonyl substituent, including 2-(benzyloxycarbonyl)ethyl and the like.

“Aminocarbonyl” refers to the group —C(O)NRR′ where each R, R′ includes independently hydrogen or C₁-C₆-alkyl or aryl or heteroaryl or “C₁-C₆-alkyl aryl” or “C₁-C₆-alkyl hetero-aryl”.

“C₁-C₆-alkyl aminocarbonyl” refers to C₁-C₆-alkyl groups having an aminocarbonyl substituent, including 2-(dimethylaminocarbonyl)ethyl and the like.

“Acylamino” refers to the group —NRC(O)R′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆alkynylheteroaryl”, “C₁-C₆-alkyl cycloalkyl”, “C₁-C₆-alkyl heterocycloalkyl”.

“C₁-C₆-alkyl acylamino” refers to C₁-C₆-alkyl groups having an acylamino substituent, including 2-(propionylamino)ethyl and the like.

“Ureido” refers to the group —NRC(O)NR′R″ where each R, R′, R″ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆alkynylheteroaryl”, “C₁-C₆-alkyl cycloalkyl”, “C₁-C₆-alkyl heterocycloalkyl”, and where R′ and R″, together with the nitrogen atom to which they are attached, can optionally form a 3-8-membered heterocycloalkyl ring.

“C₁-C₆-alkyl ureido” refers to C₁-C₆-alkyl groups having an ureido substituent, including 2-(N′-methylureido)ethyl and the like.

“Carbamate” refers to the group —NRC(O)OR′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Amino” refers to the group —NRR′ where each R,R′ is independently hydrogen or “C₁-C₆-alkyl” or “aryl” or “heteroaryl” or “C₁-C₆-alkyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

“C₁-C₆-alkyl amino” refers to C₁-C₅-alkyl groups having an amino substituent, including 2-(1-pyrrolidinyl)ethyl and the like.

“Ammonium” refers to a positively charged group —N⁺RR′R″, where each R,R′,R″ is independently “C₁-C₆-alkyl” or “C₁-C₆-alkyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

“C₁-C₆-alkyl ammonium” refers to C₁-C₆-alkyl groups having an ammonium substituent, including 2-(1-pyrrolidinyl)ethyl and the like.

“Halogen” refers to fluoro, chloro, bromo and iodo atoms.

“Sulfonyloxy” refers to a group —OSO₂—R wherein R is selected from H, “C₁-C₆-alkyl”, “C₁-C₆-alkyl” substituted with halogens, e.g., an —OSO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyloxy” refers to C₁-C₅-alkyl groups having a sulfonyloxy substituent, including 2-(methylsulfonyloxy)ethyl and the like.

“Sulfonyl” refers to group “—SO₂—R” wherein R is selected from H, “aryl”, “heteroaryl”, “C₁-C₆-alkyl”, “C₁-C₆-alkyl” substituted with halogens, e.g., an —SO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyl” refers to C₁-C₅-alkyl groups having a sulfonyl substituent, including 2-(methylsulfonyl)ethyl and the like.

“Sulfinyl” refers to a group “—S(O)—R” wherein R is selected from H, “C₁-C₆-alkyl”, “C₁-C₆-alkyl” substituted with halogens, e.g., a —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfinyl” refers to C₁-C₅-alkyl groups having a sulfinyl substituent, including 2-(methylsulfinyl)ethyl and the like.

“Sulfanyl” refers to groups —S—R where R includes H, “C₁-C₆-alkyl”, “C₁-C₆-alkyl” substituted with halogens, e.g., a —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Preferred sulfanyl groups include methylsulfanyl, ethylsulfanyl, and the like.

“C₁-C₆-alkyl sulfanyl” refers to C₁-C₅-alkyl groups having a sulfanyl substituent, including 2-(ethylsulfanyl)ethyl and the like.

“Sulfonylamino” refers to a group —NRSO₂—R′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆alkynylheteroaryl”, “C₁-C₆-alkyl cycloalkyl”, “C₁-C₆-alkyl heterocycloalkyl”.

“C₁-C₆-alkyl sulfonylamino” refers to C₁-C₅-alkyl groups having a sulfonylamino substituent, including 2-(ethylsulfonylamino)ethyl and the like.

“Aminosulfonyl” refers to a group —SO₂—NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆alkynylheteroaryl”, “C₁-C₆-alkyl cycloalkyl”, “C₁-C₆-alkyl heterocycloalkyl”.

“C₁-C₆-alkyl aminosulfonyl” refers to C₁-C₆-alkyl groups having an aminosulfonyl substituent, including 2-(cyclohexylaminosulfonyl)ethyl and the like.

“Substituted or unsubstituted”: Unless otherwise constrained by the definition of the individual substituent, the above set out groups, like “alkyl”, “alkenyl”, “alkynyl”, “aryl” and “heteroaryl” etc. groups can optionally be substituted with from 1 to 5 substituents selected from the group consisting of“C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “ammonium”, “acyl”, “acyloxy”, “acylamino”, “aminocarbonyl”, “alkoxycarbonyl”, “ureido”, “aryl”, “carbamate”, “heteroaryl”, “sulfinyl”, “sulfonyl”, “alkoxy”, “sulfanyl”, “halogen”, “carboxy”, trihalomethyl, cyano, hydroxy, mercapto, nitro, and the like. Alternatively said substitution could also comprise situations where neighbouring substituents have undergone ring closure, notably when vicinal functional substituents are involved, thus forming, e.g., lactams, lactons, cyclic anhydrides, but also acetals, thioacetals, aminals formed by ring closure for instance in an effort to obtain a protective group.

“Pharmaceutically acceptable cationic salts or complexes” is intended to define such salts as the alkali metal salts, (e.g. sodium and potassium), alkaline earth metal salts (e.g. calcium or magnesium), aluminium salts, ammonium salts and salts with organic amines such as with methylamine, dimethylamine, trimethylamine, ethylamine, triethylamine, morpholine, N—Me-D-glucamine, N,N′-bis(phenylmethyl)-1,2-ethanediamine, ethanolamine, diethanolamine, ethylenediamine, N-methylmorpholine, piperidine, benzathine (N,N′-dibenzylethylenediamine), choline, ethylene-diamine, meglumine (N-methylglucamine), benethamine (N-benzylphenethylamine), diethylamine, piperazine, thromethamine (2-amino-2-hydroxymethyl-1,3-propanediol), procaine as well as amines of formula —NR,R′,R″ wherein R, R′, R″ is independently hydrogen, alkyl or benzyl. Especially preferred salts are sodium and potassium salts.

“Pharmaceutically acceptable salts or complexes” refers to salts or complexes of the below-identified compounds of formulae (I), (Ia), (Ib), (Ic), (Id), (II) and (III) that retain the desired biological activity. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. Said compounds can also be administered as pharmaceutically acceptable quaternary salts known by a person skilled in the art, which specifically include the quarternary ammonium salt of the formula —NR,R′,R″⁺Z⁻, wherein R, R′, R″ is independently hydrogen, alkyl, or benzyl, C₁-C₆-alkyl, C₂-C₆-alkenyl, C₂-C₆-alkynyl, C₁-C₆-alkyl aryl, C₁-C₆-alkyl heteroaryl, cycloalkyl, heterocycloalkyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

“Pharmaceutically active derivative” refers to any compound that upon administration to the recipient, is capable of providing directly or indirectly, the activity disclosed herein.

“Enantiomeric excess” (ee) refers to the products that are obtained by an asymmetric synthesis, i.e. a synthesis involving non-racemic starting materials and/or reagents or a synthesis comprising at least one enantioselective step, whereby a surplus of one enantiomer in the order of at least about 52% ee is yielded.

General formula (I) according to the present invention also comprises its tautomers, its geometrical isomers, its optically active forms as enantiomers, diastereomers and its racemate forms, as well as pharmaceutically acceptable salts thereof. Preferred pharmaceutically acceptable salts of the formulae (I), (Ia), (Ib), (Ic), (Id), (II) and (III) are acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

A first aspect of the present invention consists in the use of compounds of formula (I)

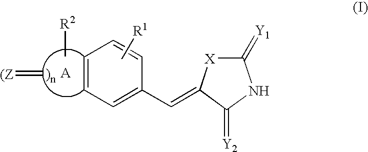

as well as its geometrical isomers, its optically active forms as enantiomers, diastereomers and its racemate forms, as well as pharmaceutically acceptable salts and pharmaceutically active derivatives thereof for the preparation of a medicament for the prophylaxis and/or treatment of autoimmune disorders and/or inflammatory diseases, cardiovascular diseases, neurodegenerative diseases, bacterial or viral infections, kidney diseases, platelet aggregation, cancer, transplantation, graft rejection or lung injuries.

In a preferred embodiment, these compounds are useful for the treatment and/or prophylaxis of autoimmune diseases or inflammatory diseases such as multiple sclerosis, psoriasis, rheumatoid arthritis, systemic lupus erythematosis, inflammatory bowel disease, lung inflammation, thrombosis or brain infection/inflammation such as meningitis or encephalitis.

In another preferred embodiment according to the invention, these compounds are useful for the treatment and/or prophylaxis of neurodegenerative diseases including multiple sclerosis, Alzheimer's disease, Huntington's disease, CNS trauma, stroke or ischemic conditions.

In a particularly preferred embodiment according to the invention, these compounds are useful for the treatment and/or prophylaxis of cardiovascular diseases such as atherosclerosis, heart hypertrophy, cardiac myocyte dysfunction, elevated blood pressure or vasoconstriction.

In another particularly preferred embodiment according to the invention, these compounds are useful for the treatment and/or prophylaxis of chronic obstructive pulmonary disease, anaphylactic shock fibrosis, psoriasis, allergic diseases, asthma, stroke or ischemic conditions, ischemia-reperfusion, platelets aggregation/activation, skeletal muscle atrophy/hypertrophy, leukocyte recruitment in cancer tissue, angiogenesis, invasion metastisis, in particular melanoma, Karposi's sarcoma, acute and chronic bacterial and viral infections, sepsis, transplantation, graft rejection, glomerulo sclerosis, glomerulo nephritis, progressive renal fibrosis, endothelial and epithelial injuries in the lung or in general lung airways inflammation.

The substituents within formula (I) are defined as follows:

A is an unsubstituted or substituted 5-8 membered heterocyclic group or an unsubstituted or substituted carbocyclic group.

Said carbocyclic group may be fused with an unsubstituted or substituted aryl, an unsubstituted or substituted heteroaryl, an unsubstituted or substituted cycloalkyl or an unsubstituted or substituted heterocycloalkyl.

Examplary heterocyclic or carbocyclic groups A include unsubstituted or substituted 2H-(benzo-1,3-dioxolanyl), unsubstituted or substituted 2H, 3H-benzo-1,4-dioxanyl, unsubstituted or substituted 2,3-dihydrobezofuranyl, unsubstituted or substituted anthraquinonyl, unsubstituted or substituted 2,2-difluorobenzo-1,3-dioxolenyl, unsubstituted or substituted 1,3-dihydrobenzofuranyl, unsubstituted or substituted benzofuranyl, unsubstituted or substituted 4-methyl-2H-benzo-1,4-oxazin-3-onyl, unsubstituted or substituted 4-methyl-2H, 3H-benzo-1,4-oxazinyl.

X is S, O or NH, preferably S.

Y¹ and Y² are independently S, O or —NH, preferably O.

Z is S or O, preferably O.

R¹ is selected from the group comprising or consisting of H, CN, carboxy, acyl, C₁-C₆-alkoxy, halogen, hydroxy, acyloxy, an unsubstituted or substituted C₁-C₆-alkyl carboxy, an unsubstituted or substituted C₁-C₆-alkyl acyloxy, an unsubstituted or substituted C₁-C₆-alkyl alkoxy, alkoxycarbonyl, an unsubstituted or substituted C₁-C₆-alkyl alkoxycarbonyl, aminocarbonyl, an unsubstituted or substituted C₁-C₆-alkyl aminocarbonyl, acylamino, an unsubstituted or substituted C₁-C₆-alkyl acylamino, ureido, an unsubstituted or substituted C₁-C₆-alkyl ureido, amino, an unsubstituted or substituted C₁-C₆-alkyl amino, ammonium, sulfonyloxy, an unsubstituted or substituted C₁-C₆-alkyl sulfonyloxy, sulfonyl, an unsubstituted or substituted C₁-C₆-alkyl sulfonyl, sulfinyl, an unsubstituted or substituted C₁-C₆-alkyl sulfinyl, sulfanyl, an unsubstituted or substituted C₁-C₆-alkyl sulfanyl, sulfonylamino, an unsubstituted or substituted C₁-C₆-alkyl sulfonylamino or carbamate. Preferably R¹ is H.

R² is selected from the group comprising or consisting of H, halogen, acyl, amino, an unsubstituted or substituted C₁-C₆-alkyl, an unsubstituted or substituted C₂-C₆-alkenyl, an unsubstituted or substituted C₂-C₆-alkynyl, an unsubstituted or substituted C₁-C₆-alkyl carboxy, an unsubstituted or substituted C₁-C₆-alkyl acyl, an unsubstituted or substituted C₁-C₆-alkyl alkoxycarbonyl, an unsubstituted or substituted C₁-C₆-alkyl aminocarbonyl, an unsubstituted or substituted C₁-C₆-alkyl acyloxy, an unsubstituted or substituted C₁-C₆-alkyl acylamino, an unsubstituted or substituted C₁-C₆-alkyl ureido, an unsubstituted or substituted C₁-C₆-alkyl carbamate, an unsubstituted or substituted C₁-C₆-alkyl amino, an unsubstituted or substituted C₁-C₆-alkyl alkoxy, an unsubstituted or substituted C₁-C₆-alkyl sulfanyl, an unsubstituted or substituted C₁-C₆-alkyl sulfinyl, an unsubstituted or substituted C₁-C₆-alkyl sulfonyl, an unsubstituted or substituted C₁-C₆-alkyl sulfonylaminoaryl, aryl, heteroaryl, an unsubstituted or substituted C₃-C₈-cycloalkyl or heterocycloalkyl, an unsubstituted or substituted C₁-C₆-alkyl aryl, an unsubstituted or substituted C₁-C₆-alkyl heteroaryl, an unsubstituted or substituted C₂-C₆-alkenyl-aryl or -heteroaryl, an unsubstituted or substituted C₂-C₆-alkynyl aryl or -heteroaryl, carboxy, cyano, hydroxy, C₁-C₆-alkoxy, nitro, acylamino, ureido, sulfonylamino, sulfanyl, or sulfonyl.

n is an integer from 0 to 2, preferably n is 0 or 1.

According to a preferred embodiment of the invention, R¹ and R² are both H.

In a particularly preferred embodiment according to the invention, X is S, Y¹ and Y² are both O, R¹ and R² are as above defined and n is 0.

A further particularly preferred aspect of the present invention is related to the use of thiazolidinedione-vinyl fused-benzene derivatives of formula (Ia), (Ib), (Ic) and (Id):

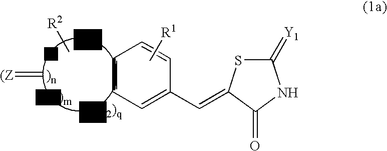

R¹, R², Y¹, Z and n in formula (Ia) are as above-defined.

G in formula (Ia) is an unsubstituted or substituted C₁-C₅ alkylene (e.g. methylene, ethylene, propylene etc.) or an unsubstituted or substituted C₁-C₅ alkenylene group (e.g. a methine (—CH═), a —CH═CH— group, a propenylene group, etc.).

W and V in formula (Ia) are each independently from each other selected from O, S, —NR³ wherein R³ is H or an unsubstituted or substituted C₁-C₆ alkyl group, m and o are each independently from each other 0 or 1, p is an integer from 1 to 4 and q is an integer from 0 to 4.

Even more preferred compounds of formula (Ia) is where G is an C₁-C₄ alkylene, thus giving compounds of formula (Ib) (i.e. p=1, 2, 3 or 4, preferably 1 or 2).

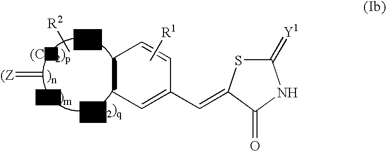

A particularly preferred sub-group of formula (Ib) are compounds having the formula (Ic), whereby W, R¹, Y¹ are as above defined and R⁶ is H or OH.

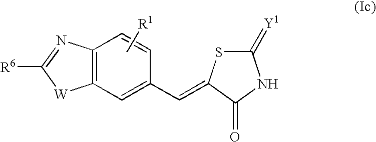

Still a further preferred sub-group of formula (Ia) are compounds, wherein V, W and Y¹ are all O, thus providing compounds of formula (Id).

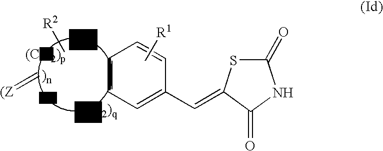

In a preferred embodiment of formulae (Ia), (Ib) or (Id), m is 0, n is 1, p is 1 or 2, q is 1, Z is O and R¹ is as above-defined.

In a further preferred embodiment of formulae (Ia), (Ib) or (Id), m is 1, n is 0, p is 1 or 2, q is 0 and R¹ and R² are as above-defined, more particularly R¹ is halogen or a hydrogen atom.

In another particularly preferred embodiment of formula (Ia), (Ib) or (Id), p is 1 or 2, q is 0, m is 0, n is 1 and R¹ and R² are as above-defined.

The compounds according to formula (I), (Ia), (Ib), (Ic), (Id), (II) and (III) are suitable for the modulation, notably the inhibition of the activity of phosphatoinositides 3-kinases (PI3K), particularly phosphatoinositides 3-kinase (PI3Kγ). It is therefore believed that the compounds of the present invention are also particularly useful for the treatment and/or prevention of disorders which are mediated by PI3Ks, particularly PI3Kγ. Said treatment involves the modulation—notably the inhibition or the down regulation—of the phosphatoinositides 3-kinases.

Compounds of the present invention include in particular those of the group consisting of:

(5Z)-5-(1,3-benzodioxol-5-ylmethylene)-1,3-thiazolidine-2,4-dione

(5Z)-5-(2,3-dihydro-1,4-benzodioxin-6-ylmethylene)-1,3-thiazolidine-2,4-dione

(5Z)-5-(2,3-dihydro-1-benzofuran-5-ylmethylene)-1,3-thiazolidine-2,4-dione

(5E)-5-[(7-methoxy-1,3-benzodioxol-5-yl)methylene]-1,3-thiazolidine-2,4-dione

(5Z)-5-[(9,10-dioxo-9,10-dihydroanthracen-2-yl)methylene]-1,3-thiazolidine-2,4-dione

(5Z)-5-[(2,2-difluoro-1,3-benzodioxol-5-yl)methylene]-1,3-thiazolidine-2,4-dione

(5Z)-5-(1,3-dihydro-2-benzofuran-5-ylmethylene)-1,3-thiazolidine-2,4-dione

(5Z)-5-(1-benzofuran-5-ylmethylene)-1,3-thiazolidine-2,4-dione

(5Z)-5-[(4-methyl-3-oxo-3,4-dihydro-2H-1,4-benzoxazin-6-yl)methylene]-1,3-thiazolidine-2,4-dione

(5Z)-5-[(4-methyl-3,4-dihydro-2H-1,4-benzoxazin-7-yl)methylene]-1,3-thiazolidine-2,4-dione

(5E)-5-(1,3-benzodioxol-5-ylmethylene)-2-thioxo-1,3-thiazolidin-4-one

(5Z)-5-(1,3-benzodioxol-5-ylmethylene)-2-imino-1,3-thiazolidin-4-one

Another aspect of the invention consists in novel thiazolidinone-vinyl fused-benzene derivatives of formula (II)

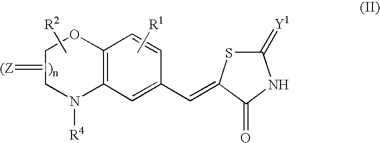

as well as its geometrical isomers, its optically active forms as enantiomers, diastereomers and its racemate forms, as well as pharmaceutically acceptable salts and pharmaceutically active derivatives thereof, wherein Y ,Z, R¹, R² are as above defined and n is 0 or 1.

R⁴ is selected in the group comprising or consisting of H, acyl, an unsubstituted or substituted C₁-C₆-alkyl, an unsubstituted or substituted C₂-C₆-alkenyl, an unsubstituted or substituted C₂-C₆-alkynyl, an unsubstituted or substituted C₁-C₆-alkyl carboxy, an unsubstituted or substituted C₁-C₆-alkyl acyl, an unsubstituted or substituted C₁-C₆-alkyl alkoxycarbonyl, an unsubstituted or substituted C₁-C₆-alkyl aminocarbonyl, an unsubstituted or substituted C₁-C₆-alkyl acyloxy, an unsubstituted or substituted C₁-C₆-alkyl, acylamino, an unsubstituted or substituted C₁-C₆-alkyl ureido, an unsubstituted or substituted C₁-C₆-alkyl amino, an unsubstituted or substituted C₁-C₆-alkyl alkoxy or an unsubstituted or substituted C₁-C₆-alkyl sulfanyl, an unsubstituted or substituted C₁-C₆alkyl sulfinyl, an unsubstituted or substituted C₁-C₆-alkyl sulfonyl, an unsubstituted or substituted C₁-C₆-alkyl sulfonylaminoaryl, an unsubstituted or substituted aryl, an unsubstituted or substituted heteroaryl, an unsubstituted or substituted C₃-C₈-cycloalkyl or heterocycloalkyl, an unsubstituted or substituted C₁-C₆-alkyl aryl, an unsubstituted or substituted C₁-C₆-alkyl heteroaryl, an unsubstituted or substituted C₂-C₆-alkenyl-aryl or -heteroaryl, an unsubstituted or substituted C₂-C₆-alkynyl aryl or -heteroaryl, carboxy, hydroxy, C₁-C₆-alkoxy, C₁-C₆ alkyl carbamate, sulfonylamino, sulfanyl or sulfonyl.

In a preferred embodiment R⁴ is an unsubstituted or substituted C₁-C₆-alkyl, an unsubstituted or substituted C₁-C₆-alkyl aryl, an unsubstituted or substituted C₁-C₆-alkyl heteroaryl, an unsubstituted or substituted aryl, an unsubstituted or substituted heteroaryl, an unsubstituted or substituted C₃-C₈-cycloalkyl or -heterocycloalkyl, an unsubstituted or substituted C₁-C₆-alkyl aryl, an unsubstituted or substituted C₁-C₆-alkyl heteroaryl, an unsubstituted or substituted C₂-C₆-alkenyl-aryl or -heteroaryl, an unsubstituted or substituted C₂-C₆-alkynyl aryl or -heteroaryl.

In another preferred embodiment according to the present invention Y¹ is O.

Still another aspect of the invention consists in novel thiazolidinone-vinyl fused-benzene derivatives of formula (III)

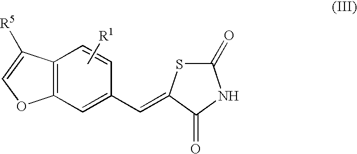

as well as its geometrical isomers, its optically active forms as enantiomers, diastereomers and its racemate forms, as well as pharmaceutically acceptable salts and pharmaceutically active derivatives thereof,

wherein R¹ is as above defined and R⁵ is selected in the group comprising or consisting of H, halogen, acyl, amino, an unsubstituted or substituted C₁-C₆-alkyl, an unsubstituted or substituted C₂-C₆-alkenyl, an unsubstituted or substituted C₂-C₆-alkynyl, an unsubstituted or substituted C₁-C₆-alkyl carboxy, an unsubstituted or substituted C₁-C₆-alkyl acyl, an unsubstituted or substituted C₁-C₆-alkyl alkoxycarbonyl, an unsubstituted or substituted C₁-C₆-alkyl aminocarbonyl, an unsubstituted or substituted C₁-C₆-alkyl acyloxy, an unsubstituted or substituted C₁-C₆-alkyl, acylamino, an unsubstituted or substituted C₁-C₆-alkyl urcido, an unsubstituted or substituted C₁-C₆-alkyl amino, an unsubstituted or substituted C₁-C₆-alkyl alkoxy or an unsubstituted or substituted C₁-C₆-alkyl sulfanyl, an unsubstituted or substituted C₁-C₆-alkyl sulfinyl, an unsubstituted or substituted C₁-C₆-alkyl sulfonyl, an unsubstituted or substituted C₁-C₆-alkyl sulfonylaminoaryl, an unsubstituted or substituted aryl, an unsubstituted or substituted heteroaryl, an unsubstituted or substituted C₃-C₈-cycloalkyl or heterocycloalkyl, an unsubstituted or substituted C₁-C₆-alkyl aryl, an unsubstituted or substituted C₁-C₆-alkyl heteroaryl, an unsubstituted or substituted C₂-C₆-alkenyl-aryl or -heteroaryl, an unsubstituted or substituted C₂-C₆-alkynyl aryl or -heteroaryl, carboxy, cyano, hydroxy, C₁-C₆-alkoxy, nitro, acylamino, C₁-C₆ alkyl carbamate, ureido, sulfonylamino, sulfanyl or sulfonyl.

A further aspect of the present invention is the use of the novel compounds of formulae (II) or (III) as medicament.

Another further aspect of the invention is a pharmaceutical composition containing at least one thiazolidinone-vinyl fused-benzene derivative according to formulae (II) or (III) and a pharmaceutically acceptable carrier, diluent or excipient thereof.

Still a further aspect of the invention is the use of compounds according to formula (II) or (III) for the preparation of a medicament for the prophylaxis and/or treatment of diseases mediated by a PI3 Kinase, particularly PI3 Kinase γ.

Specific diseases are the ones selected in the group comprising or consisting of autoimmune disorders and/or inflammatory diseases, cardiovascular diseases, neurodegenerative diseases, bacterial or viral infections, kidney diseases, platelet aggregation, cancer, transplantation, graft rejection or lung injuries.

In a preferred embodiment, said compounds are useful for the treatment and/or prophylaxis of autoimmune diseases or inflammatory diseases such as multiple sclerosis, psoriasis, rheumatoid arthritis, systemic lupus erythematosis, inflammatory bowel disease, lung inflammation, thrombosis or brain infection/inflammation such as meningitis or encephalitis.

In another preferred embodiment according to the invention, these compounds are useful for the treatment and/or prophylaxis of neurodegenerative diseases including multiple sclerosis, Alzheimer's disease, Huntington's disease, CNS trauma, stroke or ischemic conditions.

In a particularly preferred embodiment according to the invention, these compounds are useful for the treatment and/or prophylaxis of cardiovascular diseases such as atherosclerosis, heart hypertrophy, cardiac myocyte dysfunction, elevated blood pressure or vasoconstriction.

In another particularly preferred embodiment according to the invention, these compounds are useful for the treatment and/or prophylaxis of chronic obstructive pulmonary disease, anaphylactic shock fibrosis, psoriasis, allergic diseases, asthma, stroke or ischemic conditions, ischemia-reperfusion, platelets aggregation/activation, skeletal muscle atrophy/hypertrophy, leukocyte recruitment in cancer tissue, angiogenesis, invasion metastisis, in particular melanoma, Karposi's sarcoma, acute and chronic bacterial and viral infections, sepsis, transplantation, graft rejection, glomerulo sclerosis, glomerulo nephritis, progressive renal fibrosis, endothelial and epithelial injuries in the lung or in general lung airways inflammation.

According to the invention, compounds of formula (II) or (III) are suitable to modulate, particularly to inhibit, PI3 kinase activity and more particularly PI3Kγ activity.

Still a further object of the present invention is a process for preparing azolidinone-vinyl fused-benzene derivatives according to formula (I), (Ia), (Ib), (Ic) or (Id) but also thiazolidinone-vinyl fused-benzene derivatives of formulae (II) or (III).

The azolidinone-vinyl fused-benzene derivatives exemplified in this invention may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures.

In the process illustrated in the following schemes R¹, R², R⁴, R⁵, G, V, W, Y¹, Y², Z, m, n, o, p and q are each as above-defined in the description.

Generally, the azolidinone-vinyl fused-benzene derivatives according to the general formula (I) could be obtained by several synthetic approaches, using both solution-phase and solid-phase chemistry protocols (Brummond et.al., J.O.C., 64, 1723-1726 (1999)), either by convential methods or by microwave-assisted techniques.

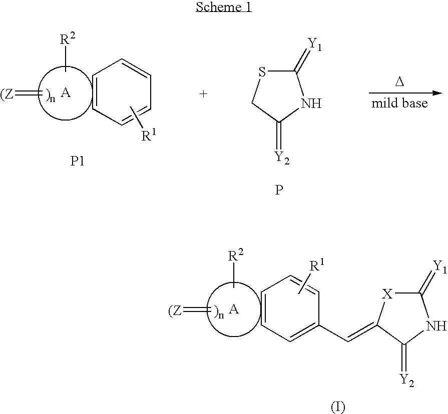

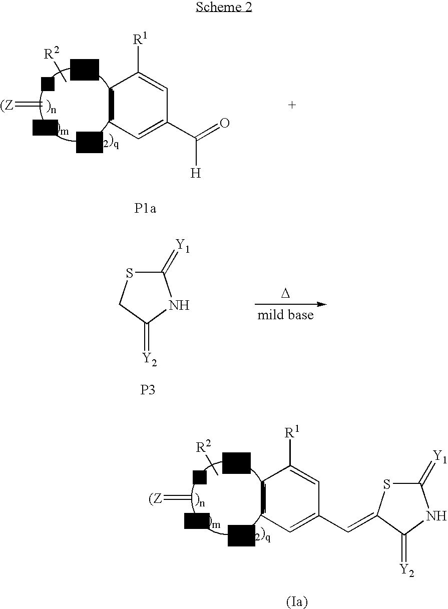

In a first step, approximately equimolar amounts of the reactant P1a and thiazolidinedione or rhodanin P3 are heated in the presence of a mild base to provide the corresponding olefin of formula (Ia). In the present step, P1a may be replaced with the following P1b and P1c in order to obtain the Formulae (Ib) and (Ic) respectively as above described in the description.

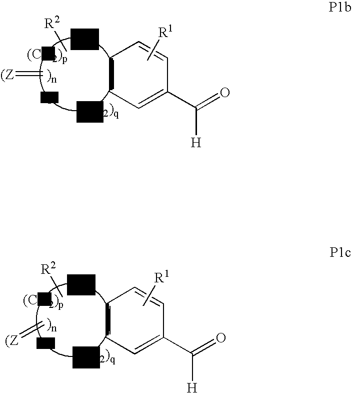

Particularly preferred process according to the invention are illustrated by the following schemes 3 and 4 in which compounds of formula (II) and (III) respectively, may be obtained using the same reaction as above-mentioned.

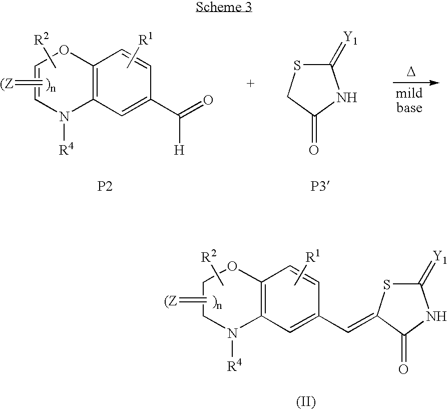

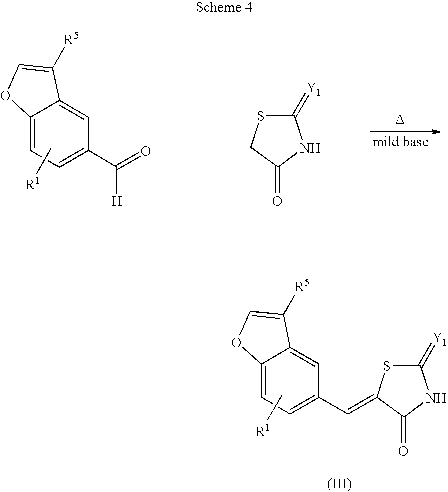

While this step may be carried out in the absence of a solvent at a temperature, which is sufficiently high to cause at least partial melting of the reaction mixture, it is preferably carried out in the presence of a reaction inert solvent. A preferred such temperature is in the range of from 100° C. to 250° C., and especially preferred is a temperature of from 120° C. to 200° C. Examples of such solvents for the above reaction include solvents like dimethoxymethane, xylene, toluene, o-dichlorobenzene etc. Examples of suitable mild bases for the above reaction are alkali metal and alkaline earth salts of week acids such as the (C₁-C₁₂)-alkyl carboxylic acids and benzoic acid, alkali metal and alkaline earth carbonates and bicarbonates such as calcium carbonate, magnesium carbonate, potassium bicarbonate and secondary amines such as piperidine, morpholine as well as tertiary amines such as pyridine, triethylamine, diisopropylethylamine, N-methylmorpholine, N-ethylpiperidine, N-methylpiperidine and the like. Especially preferred mild bases are sodium acetate or piperidine for reasons of economy and efficiency.

In a typical such reaction (Tietze et.al., in “The Knoevenagel reaction”, p.341 ff., Pergamon Press, Oxford 1991, Eds.: Trost B. M., Fleming I.) the aldehyde starting material P1a and thiazolidinedione P3 are combined in approximately equimolar amounts with 0.5 to one equivalent of piperidine in dimethoxymethane or similar solvent and heated between 120 and 200° C. at which the reaction is substantially complete in from 15 minutes to 3 hours. The desired olefin of formula (Ia) is then isolated by filtration, in case it precipitated out of the reaction mixture upon cooling, or for example, by mixing with water and subsequent filtration, to obtain the crude product, which is purified, if desired, e.g. by crystallization or by standard chromatographic methods.

Alternatively olefins of formula (Ia) may be obtained typically by mixing equimolar amounts of thiazolidinedione P3 with aldheyde P1a and molar excess, preferably a 2-4 fold excess, of anhydrous sodium acetate and the mixture is heated at a temperature high enough to effect melting, at which temperature the reaction is mainly complete in from 5 to 60 minutes. Alternatively the above reaction can be carried out in acidic media such as acetic acid in the presence of sodium acetate.

Above described reaction can be carried out alternatively under microwave conditions as heating source. Typically the aldehyde starting material P1a and thiazolidinedione P3 are combined in approximately equimolar amounts with 0.5 to one equivalent of piperidine in dimethoxymethane or similar solvent and heated between 140° C. and 240° C. at which the reaction is substantially complete in from 3 to 10 minutes.

The pharmaceutically acceptable cationic salts of compounds of the present invention are readily prepared by reacting the acid forms with an appropriate base, usually one equivalent, in a co-solvent. Typical bases are sodium hxdroxide, sodium methoxide, sodium ethoxide, sodium hydride, potassium hydroxide, potassium methoxide, magnesium hydroxide, calcium hydroxide, benzathine, choline, diethanolamine, ethylenediamine, meglumine, benethamine, diethylamine, piperazine and tromethamine. The salt is isolated by concentration to dryness or by addition of a non-solvent. In some cases, salts can be prepared by mixing a solution of the acid with a solution of the cation (sodium ethylhexanoate, magnesium oleate), employing a solvent in which the desired cationic salt precipitates, or can be otherwise isolated by concentration and addition of a non-solvent.

2,4-Azolidinone derivative P3 is commercially available from various sources. The aldehydes of formula P1a are prepared by a variety of well known methods, for example starting from the corresponding carboxylic acid alkyl ester or carboxylic acid by oxido-reduction, using standard techniques to reduce carboxylic acid alkyl ester or carboxylic acid to benzylic alcohols with Lihium aluminium hydride, Diisopropylaluminum etc. and ultimately re-oxidize the corresponding benzylic alcohol to the corresponding aldehyde by mild oxidation with reagents such as manganese dioxide, chromic acid, Dess-Martin reagent or Swern oxidation, or under conditions known to produce aldehydes from primary alcohols. An alternative way may be the direct reduction of the corresponding carboxylic acid alkyl ester or carboxylic acid to the corresponding aldehyde, using DIBAL at low temperature or any other techniques known in the field.

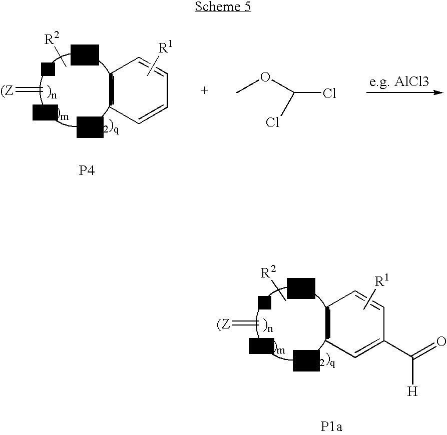

An alternative way to produce the appropriate aldehydes is the selective reduction of a nitrile moiety to the corresponding aldehyde using known methods like e.g. DIBAL etc. Another alternative way to produce the appropriate aldehydes is the reaction of the corresponding benzene derivative in a Friedl-Crafts type of reaction wherein the substrate P4 as shown in the above scheme 5 is reacted with 1,1-dichloromethylmethyl ether in the presence of a Lewis acid such as titanium tetrachloride or aluminium trichloride or any corresponding Lewis acids suitable for such type of reaction.

Acccording to a more particularly preferred process of the invention, as described in the literature (Petrov O. I., Kalcheva V. B., Antonova A. T., Collect. Czech. Chem. Commun, 62, p.494-7 (1997)) and illustrated by Scheme 6 hereinafter, reactant P2 may be obtained starting from P5 by reacting with 1,1-dichloromethylmethyl ether as above-described.

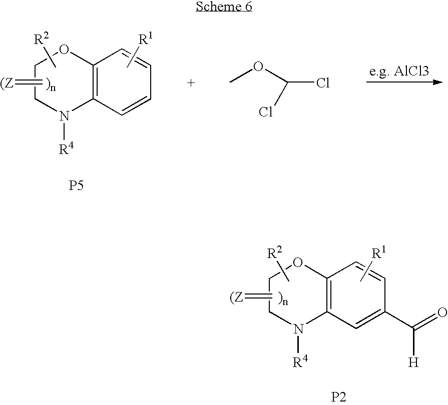

Acccording to another more particularly preferred process of the invention, as illustrated by Scheme 7 hereinafter, reactant P6 may be obtained starting from P7 by reacting with DMF and the presence of magnesium or n-butyl-lithium or any other method known to the person skilled in the art.

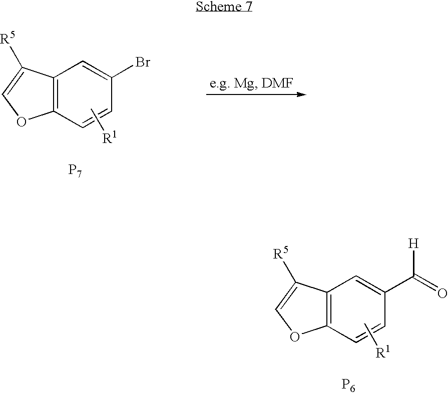

If the above set out general synthetic methods are not applicable to obtain compounds according to formula (I) and/or to necessary intermediates for the synthesis of compounds of formula (I), suitable methods of preparation known by a person skilled in the art should be used. In general, the synthesis pathways for any individual compound of formula (I) will depend on the specific substitutents of each molecule and upon the ready availability of intermediates necessary; again such factors being appreciated by those of ordinary skill in the art. For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3^{rd} Edition 1999.

Compounds of this invention can be isolated in association with solvent molecules by crys-tallization from evaporation of an appropriate solvent. The pharmaceutically acceptable acid addition salts of the compounds of formulae (I), (Ia), (Ib), (Ic), (Id), (II) and (III) which contain a basic center, may be prepared in a conventional manner. For example, a solution of the free base may be treated with a suitable acid, either neat or in a suitable solution, and the resulting salt isolated either by filtration or by evaporation under vacuum of the reaction solvent. Pharmaceutically acceptable base addition salts may be obtained in an analogous manner by treating a solu-tion of compound of formulae (I), (Ia), (Ib), (Ic), (Id), (II) and (III) with a suitable base. Both types of salts may be formed or interconverted using ion-exchange resin techniques.

When employed as pharmaceuticals, azolidinedione-vinyl fused-benzene derivatives of the present invention are typically administered in the form of a pharmaceutical composition. Hence, pharmaceutical compositions comprising a compound of formulae (I), (Ia), (Ib), (Ic), (Id), (II) and (III) and a pharmaceutically acceptable carrier, diluent or excipient therefore are also within the scope of the present invention. A person skilled in the art is aware of a whole variety of such carrier, diluent or excipient compounds suitable to formulate a pharmaceutical composition.

The compounds of the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous use). Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Pharmaceutical compositions containing azolidinedione-vinyl fused-benzene derivatives of this invention can be prepared in a manner well known in the pharmaceutical art and comprise at least one active compound. Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the present invention can be administered by a variety of routes including oral, rectal, transdermal, subcutaneous, intravenous, intramuscular and intranasal. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the thiazolidinedione-vinyl fused-benzene derivative is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dio-xide; a sweetening agent such as sucrose or saccharin; or a flavoring agent such as pepper-mint, methyl salicylate, or orange flavoring.

Injectable compositions are typically based upon injectable sterile saline or phosphate-buf-fered saline or other injectable carriers known in the art. As above mentioned, the thiazolidinedione-vinyl fused-benzene derivatives of formula (I) in such compositions is typically a minor component, frequently ranging between 0.05 to 10% by weight with the remainder being the injectable carrier and the like.

The above described components for orally administered or injectable compositions are merely representative. Further materials as well as processing techniques and the like are set out in Part 5 of Remington 's Pharmaceutical Sciences, 20^{th} Edition, 2000, Marck Publishing Company, Easton, Pa., which is incorporated herein by reference.

The compounds of this invention can also be administered in sustained release forms or from sustained release drug delivery systems. A description of representative sustained release materials can also be found in the incorporated materials in Remington's Pharmaceutical Sciences.

In the following the present invention shall be illustrated by means of some examples which are not construed to be viewed as limiting the scope of the invention. The following abbreviations are hereinafter used in the accompanying examples: min (minute), hr (hour), g (gram), mmol (millimole), m.p. (melting point), eq (equivalents), ml (milliliter), μl (microliters), ACN (acetonitrile), Boc (butoxycarbonyl), Cbz (carboxybenzyl), CDCl₃ (deuterated chloroform), cHex (cyclohexane), dba (dibenzylideneacetone), DCM (dichloromethane), DEAD (diethylazodicarboxylate, DIC (diisopropylcarbodiimide), DIEA (diisopropylethylamine), DMAP (4-dimethylaminopyridine), DME (dimethoxyethane), DMF (dimethylformamide), DMSO (dimethylsulfoxide), DMSO-d₆ (deuterated dimethylsulfoxide), EDC (1-(3-dimethylaminopropyl)-3-ethylcarbodiimide hydrochloride), EtOAc (ethyl acetate), Et₂O (diethylether), Fmoc (9-fluorenylmethoxy-carbonyl), HOBt (1-hydroxybenzotriazole), K₂CO₃ (potassium carbonate), MgSO₄ (magnesium sulfate), MsCl (methylsulfonylchloride), MTBE (tert-butylmethylether), NaH (sodium hydride), NaHCO₃ (sodium bicarbonate), nBuLi (n-butyllithium), PCC (pyridinium chlorochromate), PE (petroleum ether), QCl (tetrabutylammonium chloride), rt (room temperature), TBTU (O-benzotriazolyl-N,N,N′,N′-tetramethyluronium-tetrafluoroborate), TEA (triethylamine), TFA (trifluoroacetic acid), THF (tetrahydrofuran), TMOF (trimethylorthoformate), TMAD (N,N,N′,N′-tetramethylazodicarboxamide), TosCl (to luenesul fonyl chloride).

### EXAMPLES

The following list of compounds were synthesized according to the below mentioned methods:

(5Z)-5-(1,3-benzodioxol-5-ylmethylene)-1,3-thiazolidine-2,4-dione

(5E)-5-(1,3-benzodioxol-5-ylmethylene)-2-thioxo-1,3-thiazolidin-4-one

(5Z)-5-(2,3-dihydro-1,4-benzodioxin-6-ylmethylene)-1,3-thiazolidine-2,4-dione

(5Z)-5-(2,3-dihydro-1-benzofuran-5-ylmethylene)-1,3-thiazolidine-2,4-dione

(5E)-5-[(7-methoxy-1,3-benzodioxol-5-yl)methylene]-1,3-thiazolidine-2,4-dione

(5Z)-5-[(9,10-dioxo-9,10-dihydroanthracen-2-yl)methylene]-1,3-thiazolidine-2,4-dione

(5Z)-5-[(2,2-difluoro-1,3-benzodioxol-5-yl)methylene]-1,3-thiazolidine-2,4-dione

(5Z)-5-(1,3-dihydro-2-benzofuran-5-ylmethylene)-1,3-thiazolidine-2,4-dione

(5Z)-5-(1-benzofuran-5-ylmethylene)-1,3-thiazolidine-2,4-dione

(5Z)-5-[(4-methyl-3-oxo-3,4-dihydro-2H-1,4-benzoxazin-6-yl)methylene]-1,3-thiazolidene-2,4-dione

(5Z)-5-[(4-methyl-3,4-dihydro-2H-1,4-benzoxazin-7-yl)methylene]-1,3-thiazolidine-2,4-dione

(5Z)-5-(1,3-benzodioxol-5-ylmethylene)-2-imino-1,3-thiazolidin-4-one

The following intermediate aldehydes are commercially available: 2,2-Difluoro-1,3-benzodioxole-5-carboxaldehyde, 1,3-Benzodioxole-5-carboxaldehyde, 1,4-Benzodioxan-6-carboxaldehyde, 9,10-Dioxo-9,10-dihydro-anthracene-2-carbaldehyde, 2,3-Dihydro-benzo[b]furan-5-carboxaldehyde, 3-Methoxy-4,5-methylenedioxybenzaldehyde.

Thiazolidinedione and Rhodanine are commercially available. Intermediate aldehydes, 5-Formyl-1-benzofuran, 4-Methyl-3-oxo-3,4-dihydro-2H-benzo[1,4]oxazine-6-carbaldehyde, 4-Methyl-3,4-dihydro-2H-benzo[1,4]oxazine-7-carbaldehyde and 1,3-Dihydroisobenzofuran-5-carbaldehyde, were synthesized according to the protocols as mentioned below.

The HPLC, NMR and MS data provided in the examples described below were obtained as followed: HPLC: column Waters Symmetry C8 50×4.6 mm, Conditions: MeCN/H₂O, 5 to 100% (8 min), max plot 230-400 nm; Mass spectra: PE-SCIEX API 150 EX (APCI and ESI), LC/MS spectra: Waters ZMD (ES); ¹H-NMR: Bruker DPX-300 MHz.

The purifications were obtained as followed: Preparative HPLC Waters Prep LC 4000 System equipped with columns Prep Nova-Pak®HR C186 μm 60 Å, 40×30 mm (up to 100 mg) or 40×300 mm (up to 1g). All the purifications were performed with a gradient of MeCN/H₂O 0.09% TFA.

### Intermediate 1

### Preparation of 5-formyl-1-benzofuran

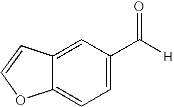

### Step I

### Ethyl-2-formyl-4-bromophenoxy acetate

A mixture of 5-bromosalicylaldehyde (50 g, 0.248 mol), ethylbromoacetate (42 g, 0.248 mol) and K₂CO₃ (68 g, 0.49 mol) in dry DMF (200 mL) was stirred at RT for 12 h. The reaction mixture was filtered and filtrate diluted with water. The mixture was extracted with diethylether (4×200 mL), washed with brine and concentrated to give crude ethyl-2-formyl-4-bromophenoxy acetate (64 g, 90%) as a solid.

### Step II

### 4-Bromo-2-formylphenoxy acetic acid

A mixture of ethyl-2-formyl-4-bromophenoxy acetate (60 g, 0.209 mol), LiOH (7.5 g, 0.31 mol), THF (250 mL) and water (100 mL) was stirred at RT for 24 h. The reaction mixture was concentrated under reduce pressure and residue acidified with 1.5N HCl to pH=2. The solid precipitate obtained was filtered and dried to give 4-bromo-2-formylphenoxy acetic acid (50 g, 94%).

### Step III

### 5-Bromo-1-benzofuran

To a mixture of 2-formyl-4-bromophenoxy acetic acid (50 g, 0.192 mol), sodium acetate (100 g, 1.21 mol) in acetic acid (250 mL) at 100° C. was added acetic anhydride (100 mL) portions during a period of 3 h. The reaction mixture was then refluxed for 20 h. The solvent was removed by distillation and residue diluted with 3N HCl (500 mL) and refluxed for 2 h. The reaction mixture was then concentrated under vacuum and product extracted with pet. ether (3×200 mL). The organic layer was washed with 10% NaHCO₃ solution and evaporated to give 5-bromo-1-benzofuran (15 g, 40%) as a pale yellow liquid.

### Step IV

### 5-Formyl-1-benzofuran (P1a in scheme 2 for example 9)

A mixture of 5-bromo-1-benzofuran (0.5 g), Mg (0.92 g, 0.038 mol), I₂ (1 crystal) in dry THF (2.5 mL) under N₂ atmosphere was refluxed for 30 min. To this was added a solution of 5-bromo-1-benzofuran (4.5 g) in 25 mL of dry THF) as soon as the I₂ color disappear and refluxed for another 2 h. The reaction mixture was then cooled to 40° C. and added dry DMF (3.6 g) drop-wise and slowly warmed to RT for a period of 12 h. The reaction mixture was then cooled to 0° C. and acidified with 3N HCl to pH=2 and stirred for 30 min. The reaction mixture was then diluted with water (500 mL), extracted with ethylacetate (2×200 mL), washed with brine and dried. The solvent was removed under vacuum and purified by column chromatography over silica gel (pet. ether/CH₂Cl₂) to give 5-formyl-1-benzofuran (2 g, 54%) as a liquid. LC-MS: M/Z ESI: 1.47 min, 147.34 (M+1).

### Intermediate 2

### Preparation of 4-Methyl-3-oxo-3,4-dihydro-2H-benzo[1,4]oxazine-6-carbaldehyde

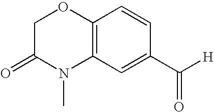

### Step I

### 2-(N-methylamino)-phenol

1 g of benzoxazole was dissolved in 20 ml of THF. 0.9 g of NaBH₄ were added under nitrogen and stirring. The suspension was cooled to 0° C. and 0.86 ml of acetic acid dissolved in 5 ml THF were slowly added, keeping the reaction temperature below 5° C. The reaction was stirred at 0° C. for 30 minutes and for further 12 hours at room temperature. The reaction mixture was again cooled to 0° C. and 50 ml of sat. NH₄Cl solution were added_carefully. The phases were separated and the aqueous layer extracted twice with EtOAc. The combined organic layers were washed with brine, dried over MgSO4 and filtered. Removal of the solvent afforded 0.97 g (of pure 2-(N-methylamino)-phenol.

### Step II

### 4-Methyl-4H-benzo[1,4]oxazin-3-one

1 g of 2-(N-methylamino)-phenol were dissolved in chloroform, followed by the addition of 10 ml of sat. NaHCO₃ in water. To this suspension was added slowly under vigorous stirring a solution of 1 g of 2-chloroacetylchloride in acetone. The reaction mixture was stirred for 2 hours at room temperature. The layers were separated. The organic layer was washed with water and dried over Na₂SO₄. After evaporating the solvent, the red oil was taken up in 30 ml DMF and 1 g of K₂CO₃ were added and the slurry was heated at 70° C. for additional 2 hours. The cyclization was followed by TLC. 200 ml of EtOAc were added and the organic layer was washed 3× with 0.1N HCl and 5× with brine. The remaining organic layer was dried over MgSO4 and filtrated. EtOAc was removed under reduced pressure affording 1.45 g of pure 4-methyl-4H-benzo[1,4]oxazin-3-one.

### Step III

### 4-Methyl-3-oxo-3,4-dihydro-2H-benzo[1,4]oxazine-6-carbaldehyde (compound P1a of scheme 2, for use in the preparation of the compound of example 10 below)

1 g of AlCl₃ were suspended in 10 ml DCM, 0.5 ml of nitromethane were added to dissolve AlCl₃, and the solution was cooled to 0° C. 4-Methyl-4H-benzo[1,4]oxazin-3-one (0.5 g, 3.06 mmol) dissolved in DCM was added to the above solution and stirred for 15 minutes at 0° C. To this solution was further added 0.36 ml of bis-chloromethylmethylether in DCM. The reaction was stirred at 0° C. for 15 minutes and at room temperature for 3 h. The crude reaction mixture was then poured onto ice, the layers were separated and the organic phase was washed with NaHCO₃ and brine. After drying over MgSO₄ and filtration the solvent was evaporated, which afforded 0.43 g of crude product. The dark oil was purified by flash chromatography using EtOAc and cyclohexane as eluents, affording 0.2 g (37%) of 4-methyl-3-oxo-3,4-dihydro-2H-benzo[1,4]oxazine-6-carbaldehyde as colourless solid.

HPLC: 2.07 min. LC-MS: M/Z ESI: 1.31 min, 192.28 (M+1).

### Intermediate 3

### Preparation of 4-methyl-3,4-dihydro-2H-benzo[1,4]oxazine-7-carbaldehyde

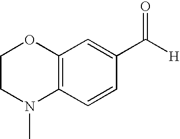

### Step I

### 4-Methyl-3,4-dihydro-2H-benzo[1,4]oxazine

0.97 g of 2-(N-methylamino)-phenol were dissolved in 50 ml acetone, followed by the addition of 2 g of K₂CO₃ dissolved in water. To this suspension was added slowly a solution of 2.66 g of dibromoethane in acetone. The reaction mixture was stirred for 22 hours under reflux. Acetone was evaporated and 200 ml of EtOAc were added and the organic layer was washed 3× with 0.1N HCl and 3× with brine. The remaining organic layer was dried over MgSO4 and filtrated. EtOAc was removed under reduced pressure affording 1 g of pure 4-methyl-3,4-dihydro-2H-benzo[1,4]oxazine.

### Step II

### 4-Methyl-3,4-dihydro-2H-benzo[1,4]oxazine-7-carbaldehyde (compound P1a of scheme 2, for use in the preparation of the compound of example 11 below)

4-Methyl-3,4-dihydro-2H-benzo[1,4]oxazine dissolved in 200 ul DMF under Argon. POCl₃ was added under Argon. The reaction was heated and a closed vial at 90° C. for 75 min. 1 ml of NaAc in water was added and stirred while a brown oil was formed. The oil was extracted with DCM. The organic layer was washed with brine, dried and evaporated to dryness, affording 0.18 g (76%) of 4-methyl-3,4-dihydro-2H-benzo[1,4]oxazine-7-carbaldehyde as colourless solid.

LC-MS: M/Z ESI: 1.37 min, 178.35 (M+1).

### Intermediate 4

### Preparation of 1,3-Dihydroisobenzofuran-5-carbaldehyde

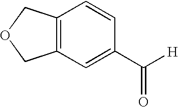

### Step I

### (1,3-Dihydro-isobenzofuran-5-yl)-methanol

In a round bottom flask with reflux condenser were placed 1.0 g of 3-Prop-2-ynyloxy-propyne and 2.08 g of propargylic alcohol in 10 ml ethanol, followed by the addition of 9.8 mg of tris(triphenylphosphine)rhodium chloride (Wilkinson catalyst) at room temperature. The reaction was heated up to 70° C., while the reaction colour turned yellow rapidly. After 1 day stirring at r.t., TLC analysis showed complete conversion of the starting material. The solvent was evaporated, diluted with DCM and extracted with H₂O, dried over MgSO4. The brown mixture was purified by flash chromatography using 8/2 cyclohexane/AcOEt as mobile phase affording (1,3-Dihydro-isobenzofuran-5-yl)-methanol as a colourless pure solid (0.92 g, 60%).

### Step II

### 1,3-Dihydroisobenzofuran-5-carbaldehyde (P1a in scheme 2 for example 8)

(1,3-Dihydro-isobenzofuran-5-yl)-methanol (440 mg, 2.9 mmol) was dissolved in 20 ml of DCM. 1,1,1-Triacetoxy-1,1-dihydro-1,2-benziodoxol-3(1H)-one (Dess-Martin reagent) (1.3 g, 3.2 mmol) was added and the reaction was stirred at r.t. for 4 h. The reaction mixture was diluted with ether and extracted 2× with NaOH 1N, 2× with H₂O and dried over MgSO₄. The crude product was sufficiently pure and used without any further purification. HPLC: 2.00 min. LC-MS: M/Z ESI: 1.50 min, 149.18 (M+1).

### Example 1

### Preparation of (5Z)-5-(1,3-benzodioxol-5-ylmethylene)-1,3-thiazolidine-2,4-dione

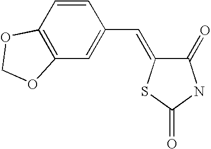

In a 100 ml round bottom flask were placed 3.9 g of thiazolidine, 5 g of piperonal and 1.65 ml of piperidine in 50 ml of DME. The reaction was stirred for 3 h at 120° C. and then slowly cooled to room temperature, while the desired condensation product crystallized. The crystals were filtered, washed with DME (rt.) and than recrystallized from DME (25 ml), affording 3.2 g of pure (5Z)-5-(1,3-benzodioxol-5-ylmethylene)-1,3-thiazolidine-2,4-dione. The corresponding potassium salt was obtained via the following route: (5Z)-5-(1,3-benzodioxol-5-ylmethylene)-1,3-thiazolidene-2,4-dione. was suspended in THF, followed by the addition of 1N solution of KOH in water (1.0 eq.). A clear solution has been obtained, which upon lyophilization gave pure potassium salt of (5Z)-5-(1,3-benzodioxol-5-ylmethylene)-1,3-thiazolidine-2,4-dione.

HPLC: 3.48 min. LC-MS: M/Z ESI: 1.31 min, 248.12 (M−1). NMR (parent): ¹H NMR (DMSO-d6) δ12.5 (br. s, 1H), 7.71 (s, 1H), 7.06-7.16 (m, 3H), 6.12 (s, 2H).

In cases were the final compounds did not crystallize from the reaction solutions, small quantities of water were added, leading to the precipitation of the desired condensation product.

The crude was either recrystallized from an appropriate solvent like DME, methanol, EtOAc or purified by flash-chromatography using EtOAc, cyclohexane mixtures as eluents.

Alternatively the final compounds could be synthesized in a parallel manner according to the following protocol:

In a parallel synthesizer Quest 210™ was placed the corresponding aldehyde, to which was added a mixture of piperidine (17.9 mg/tube) and 2,4-thiazolidinedione (49.2 mg/tube) in DME (2 ml/tube). The reactions were stirred for 3 h at 120° C. and then cooled to room temperature under agitation. 2 ml of H₂O were added. Those compounds, which precipitated were filtered off via the lower manifold. The remaining clear solutions were reduced in volume, followed by the addition of water. The so formed solids were filtered and washed with little amount of DME, affording pure condensation products.

### Example 2

### Preparation of (5E)-5-(1,3-benzodioxol-5-ylmethylene)-2-thioxo-1,3-thiazolidin-4-one

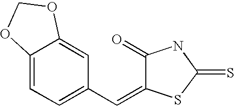

In a 24 ml vial was placed 1 g of commercially available rhodanine, 1.3 g of piperonal and 0.5 ml of TEA in 10 ml of DME. The reaction was stirred for 5 h at 120° C. and then cooled to room temperature upon which the final product precipitated. The solid was filtered and washed with DME affording 1.6 g (80%) of orange powder.

LC-MS: M/Z ESI: 1.46 min, 266.00 (M+1), 264.08 (M−1). NMR (parent): ¹H NMR (DMSO-d6) δ13.75 (br. s, 1H), 7.58 (s, 1H), 7.08-7.18 (m, 3H), 6.14 (s, 2H).

### Example 3

### Preparation of (5Z)-5-(2,3-dihydro-1,4-benzodioxin-6-ylmethylene)-1,3-thiazolidine-2,4-dione

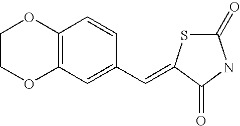

Following the general method as outlined in Example 1, starting from 2,3-dihydro-1,4-benzodioxin-6-carbaldehyde and 1,3-thiazolidine-2,4-dione, the title compound was obtained.

264 (M+1), 262 (M−1). ¹H NMR: (DMSO-d6) δ12.52 (br. s, 1H), 7.68 (s, 1H,), 7.09 (dd, 2H, J=1.9, 7.1), 7.00 (d, 1H, J=9.0 Hz), 4.36-4.22 (m, 4H).

### Example 4

### Preparation of (5Z)-5-(2,3-dihydro-1-benzofuran-5-ylmethylene-1,3-thiazolidine-2,4-dione

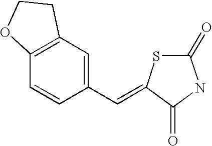

Following the general method as outlined in Example 1, starting from 2,3-dihydro-1-benzofuran-5-carbaldehyde and 1,3-thiazolidine-2,4-dione, the title compound was obtained.

248 (M+1), 246 (M−1). ¹H NMR: (DMSO-d6) δ9.80 (br. s, 1H), 7.37 (s, 1H,), 7.25 (d, 1H, J=8.3),7.21 (s, 1H), 6.80 (d, 1H, J=8.3 Hz), 4.54 (t, 2H, J=8.85), 3.19 (t, 2H, J=8.85)

### Example 5

### Preparation of (5E)-5-[(7-methoxy-1,3-benzodioxol-5-yl)methylene]-1,3-thiazolidine-2,4-dione

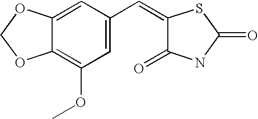

Following the general method as outlined in Example 1, starting from 7-methoxy-1,3-benzodioxol-5-yl)carbaldehyde and 1,3-thiazolidine-2,4-dione, the title compound was obtained.

280 (M+1), 278 (M−1). ¹H NMR: (DMSO-d6) δ12.63 (br. s, 1H), 7.78 (s, 1H,), 7.65 (s, 1H), 7.57 (d, 1H, J=8.5Hz), 7.45 (dd, 2H, J=0.8, 7.6).

### Example 6

### Preparation of (5Z)-5-[(9,10-dioxo-9,10-dihydroanthracen-2-yl)methylene]-1,3-thiazolidene-2,4-dione

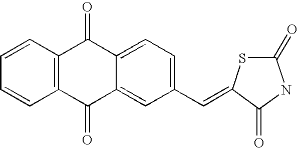

Following the general method as outlined in Example 1, starting from (9,10-dioxo-9,10-dihydroanthracen-2-yl)carbaldehyde and 1,3-thiazolidine-2,4-dione, the title compound was obtained.

336 (M+1), 334 (M−1).

### Example 7

### Preparation of (5Z)-5-[(2,2-difluoro-1,3-benzodioxol-5-yl)methylene]-1,3-thiazolidine-2,4-dione

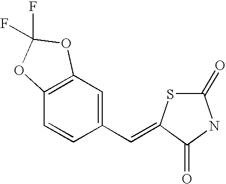

Following the general method as outlined in Example 1, starting from (2,2-difluoro-1,3-benzodioxol-5-yl)carbaldehyde and 1,3-thiazolidine-2,4-dione, the title compound was obtained.

286 (M+1), 284 (M−1). ¹H NMR: (DMSO-d6) δ12.63 (br. s, 1H), 7.78 (s, 1H,), 7.65 (s, 1H), 7.57 (d, 1H, J 8.5 Hz), 7.45 (dd, 2H, J=0.8, 7.6)

### Example 8

### Preparation of (5Z)-5-(1,3-dihydro-2-benzofuran-5-ylmethylene)-1,3-thiazolidine-2,4-dione

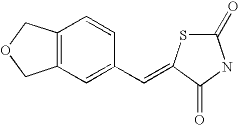

Following the general method as outlined in Example 1, starting from 1,3-dihydro-2-benzofuran-5-carbaldehyde and 1,3-thiazolidine-2,4-dione, the title compound was obtained.

248 (M+1), 246 (M−1). ¹H NMR: (DMSO-d6) δ12.60 (br. s, 1H), 7.80 (s, 1H,), 7.56-7.42 (m, 2H), 5.03 (s, 4H)

### Example 9

### Preparation of (5Z)-5-(1-benzofuran-5-ylmethylene)-1,3-thiazolidine-2,4-dione

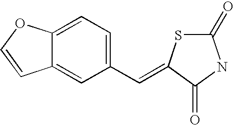

Following the general method as outlined in Example 1, starting from 1-benzofuran-5-carbaldehyde and 1,3-thiazolidine-2,4-dione, the title compound was obtained.

264 (M+1), 244 (M−1). ¹H NMR: (DMSO-d6) δ12.58 (br. s, 1H), 8.10 (d, 1H, J=2.2 Hz), 7.92 (s, 2H), 7.74 (d, 1H, J=8.6 Hz), 7.57 (d, 1H, J=8.6 Hz), 7.07 (s, 1H)

### Example 10

### Preparation of (5Z)-5-[(4-methyl-3-oxo-3,4-dihydro-2H-1,4-benzoxazin-6-yl)methylene]-1,3-thiazolidine-2,4-dione

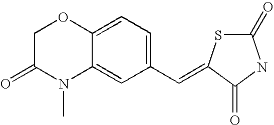

Following the general method as outlined in Example 1, starting from [(4-methyl-3-oxo-3,4-dihydro-2H-1,4-benzoxazin-6-yl)carbalehyde and 1,3-thiazolidine-2,4-dione, the title compound was obtained.

291 (M+1), 289 (M−1). ¹H NMR: (DMSO-d6) δ12.58 (br. s, 1H), 7.81 (s, 1H), 7.41 (s, 1H), 7.13-7.26 (d, 2H), 4.74 (s, 2H), 2.99 (s, 3H)

### Example 11

### Preparation of (5Z)-5-[(4-methyl-3,4-dihydro-2H-1,4-benzoxazin-7yl)methylene]-1,3-thiazolidene-2,4-dione

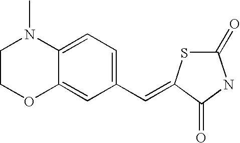

Following the general method as outlined in Example 1, starting from [(4-methyl-3,4-dihydro-2H-1,4-benzoxazin-7-yl)methylene and 1,3-thiazolidine-2,4-dione, the title compound was obtained.

277 (M+1), 275 (M−1). ¹H NMR: (DMSO-d6) δ12.34 (br. s, 1H), 7.60 (s, 1H), 7.08 (d, 1H, J=8.5 Hz), 6.88 (s, 1H), 6.79 (d, 1H, J=8.5 Hz), 4.21 (m, 2H), 3.41 (m, 2H), 2.94 (s, 3H).

### Example 12

### Preparation of (5Z)-5-(1,3-benzodioxol-5-ylmethylene)-2-imino-1,3-thiazolidin-4-one

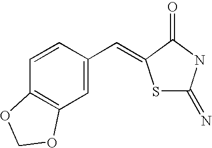

Following the general method as outlined in Example 1, starting from 1,3-benzodioxol-5-carbaldehyde and 2-imino-1,3-thiazolidin-4-one, the title compound was obtained.

249 (M+1), 247 (M−1). ¹H NMR: (DMSO-d6) δ

### Example 13

### Preparation of a Pharmaceutical Formulation

The following formulation examples illustrate representative pharmaceutical compositions according to the present invention being not restricted thereto.

### Formulation 1

### Tablets

A compound of formula (I) is admixed as a dry powder with a dry gelatin binder in an approximate 1:2 weight ration. A minor amount of magnesium stearate is added as a lubricant. The mixture is formed into 240-270 mg tablets (80-90 mg) of active azolidinone compound per tablet) in a tablet press.

### Formulation 2

### Capsules

A compound of formula (I) is admixed as a dry powder with a starch diluent in an approximate 1:1 weight ratio. The mixture is filled into 250 mg capsules (125 mg of active azolidinone compound per capsule).

### Formulation 3

### Liquid

A compound of formula (I) (1250 mg), sucrose (1.75 g) and xanthan gum (4 mg) are blended, passed through a No. 10 mesh U.S. sieve, and then mixed with a previously prepared solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

### Formulation 4

### Tablets

A compound of formula (I) is admixed as a dry powder with a dry gelatin binder in an approximate 1:2 weight ratio. A minor amount of magnesium stearate is added as a lubricant. The mixture is formed into 450-900 mg tablets (150-300 mg of active azolidinone compound) in a tablet press.

### Formulation 5

### Injection

A compound of formula (I) is dissolved in a buffered sterile saline injectable aqueous medium to a concentration of approximately 5 mg/ml.

### Example 14

### Biological Assays

The compounds of the present invention may be subjected to the following assays:

### a) High Throughput PI3K Lipid Kinase Assay (Binding Assay)

The assay combines the scintillation proximity assay technology (SPA, Amersham) with the capacity of neomycin (a polycationic antibiotic) to bind phospholipids with high affinity and specificity. The Scintillation Proximity Assay is based on the properties of weakly emitting isotopes (such as ³H, ¹²⁵I, ³³P). Coating SPA beads with neomycin allows the detection of phosphorylated lipid substrates after incubation with recombinant PI3K and radioactive ATP in the same well, by capturing the radioactive phospholipids to the SPA beads through their specific binding to neomycin.

To a 384 wells MTP containing 5 μl of a chemical compound library (containing 6% DMSO), the following assay components are added. 1) 5 μl (58 ng) of Human recombinant GST-PI3Kγ (in Hepes 40 mM, pH 7.4, DTT 1 mM and ethylenglycol 5%) 2) 10 μl of lipid micelles and 3) 10 μl of Kinase buffer ([³³P]γ-ATP 45 μM/60 nCi, MgCl₂ 30 mM, DTT 1 mM, P-Glycerophosphate 1 mM, NaVO₄ 100 μM, Na Cholate 0.3%, in Hepes 40 mM, pH 7.4). After incubation at room temperature for 180 minutes, with gentle agitation, the reaction is stopped by addition of 60 μl of a solution containing 100 μg of neomycin-coated PVT SPA beads in PBS containing ATP 10 mM and EDTA 5 mM. The assay is further incubated at room temperature for 60 minutes with gentle agitation to allow binding of phospholipids to neomycin-SPA beads. After precipitation of the neomycin-coated PVT SPA beads for 5 minutes at 1500×g, radioactive PtdIns(3)P is quantified by scintillation counting in a Wallac MicroBeta™ plate counter.

The values indicated in respect of PI3K γ refer to the IC₅₀ (μM), i.e. the amount necessary to achieve 50% inhibition of said target. Said values show a considerable potency of the azolidinone-vinyl fused-benzene compounds with regard to PI3Kγ.

The tested compounds according to formula (I) display an inhibition (IC₅₀) with regard to PI3Kγ of less than 2 μM, more preferred equal or less than 1 μM.

Examples of inhibitory activities for test compounds 1, 2 and 10 as set out in Table 1.

TABLE 1
IC₅₀ values of azolidinone-vinyl fused-benzene
derivatives against PI3Kγ.

| Example No | PI3Kγ, IC₅₀ (μM) |
|---|---|
| 1 | 0.05 |
| 2 | 0.06 |
| 10 | 0.03 |

### b) Cell Based ELISA to Monitor PI3K Inhibition

Measurement of Akt/PKB phosphorylation in macrophages after stimulation with C5a: Raw 264: Raw 264-7 macrophages (cultured in DMEM-F12 medium containing 10%

Fetal Calf serum and antibiotics) are plated at 20'000 cells/well in a 96 MTP 24 h before cell stimulation. Previous to the stimulation with 50 nM of Complement 5a during 5 minutes, Cells are serum starved for 2 h, and pretreated with inhibitors for 20 minutes. After stimulation cells are fixed in 4% formaldehyde for 20 minutes and washed 3 times in PBS containing 1% Triton X-100 (PBS/Triton). Endogenous peroxidase is blocked by a 20 minutes incubation in 0.6% H₂O₂ and 0.1% Sodium Azide in PBS/Triton and washed 3 times in PBS/Triton. Cells are then blocked by 60 minutes incubation with 10% fetal calf serum in PBS/Triton. Next, phosphorylated Akt/PKB is detected by an overnight incubation at 4° C. with first antibody (anti phospho Serine 473 Akt IHC, Cell Signaling) diluted 800-fold in PBS/Triton, containing 5% bovine serum albumin (BSA). After 3 washes in PBS/Triton, cells are incubated for 60 minutes with a peroxidase conjugated goat-anti-rabbit antibody (1/400 dilution in PBS/Triton, containing 5% BSA), washed 3 times in PBS/Triton, and 2 times in PBS and further incubated in 100 μl of substrate reagent solution (R&D) for 20 minutes. The reaction is stopped by addition of 50 μI of 1 M SO₄H₂ and absorbance is read at 450 nm.

The values indicated reflect the percentage of inhibition of AKT phoshorylation as compared to basal level. Said values show a clear effect of the azolidinone-vinyl fused-benzene compounds on the activation of AKT phosphorylation in macrophages.

Compounds of examples 9 and 10, when used at 10 μM completely inhibit C5a-mediated AKT phosophorylation. Examples 1, 2 or 4, when used at 10 μM, inhibit 80% of the C5a-mediated AKT-phosphorylation.

1. Use of a compound according to formula (I) 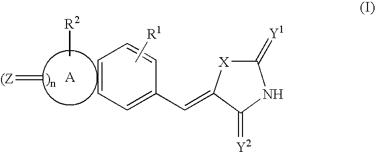
as well as its geometrical isomers, its optically active forms as enantiomers, diastereo-mers and its racemate forms, as well as pharmaceutically acceptable salts and pharma-ceutically active derivatives thereof, wherein
A is a 5-8 membered heterocyclic or carbocyclic group, wherein said carbocyclic group may be fused with aryl, heteroaryl, cycloalkyl or heterocycloalkyl;
X is S, O or NH;
Y¹ and Y² are independently S, O or —NH;
Z is S or O;
R¹ is H, CN, carboxy, acyl, C₁-C₆-alkoxy, halogen, hydroxy, acyloxy, C₁-C₆-alkyl carboxy, C₁-C₆-alkyl acyloxy, C₁-C₆-alkyl alkoxy, alkoxycarbony, C₁-C₆-alkyl alkoxycarbonyl, aminocarbonyl, C₁-C₆-alkyl aminocarbonyl, acylamino, C₁-C₆-alkyl acylamino, ureido, C₁-C₆-alkyl ureido, amino, C₁-C₆-alkyl amino, ammonium, sulfonyloxy, C₁-C₆-alkyl sulfonyloxy, sulfonyl, C₁-C₆-alkyl sulfonyl, sulfinyl, C₁-C₆-alkyl sulfinyl, sulfanyl, C₁-C₆-alkyl sulfanyl, sulfonylamino, C₁-C₆-alkyl sulfonylamino or carbamate;
R² is selected from the group comprising or consisting of H, halogen, acyl, amino, C₁-C₆-alkyl, C₂-C₆-alkenyl, C₂-C₆-alkynyl, C₁-C₆-alkyl carboxy, C₁-C₆-alkyl acyl, C₁-C₆-alkyl alkoxycarbonyl, C₁-C₆-alkyl aminocarbonyl, C₁-C₆-alkyl acyloxy, C₁-C₆-alkyl acylamino, C₁-C₆-alkyl ureido, C₁-C₆-alkyl amino, C₁-C₆-alkyl alkoxy, C₁-C₆-alkyl sulfanyl, C₁-C₆-alkyl sulfinyl, C₁-C₆-alkyl sulfonyl, C₁-C₆-alkyl sulfonylaminoaryl, aryl, heteroaryl, C₃-C₈-cycloalkyl or heterocycloalkyl, C₁-C₆-alkyl aryl, C₁-C₆-alkyl heteroaryl, C₂-C₆-alkenyl-aryl or -heteroaryl, C₂-C₆-alkynyl aryl or -heteroaryl, carboxy, cyano, hydroxy, C₁-C₆-alkoxy, nitro, acylamino, ureido, C₁-C₆-alkyl carbamate, sulfonylamino, sulfanyl, or sulfonyl;
n is 0, 1 or 2;
for the preparation of a medicament for the prophylaxis and/or treatment of autoimmune disorders and/or inflammatory diseases, cardiovascular diseases, neurodegenerative diseases, bacterial or viral infections, kidney diseases, platelet aggregation, cancer, transplantation, graft rejection or lung injuries.

2. Use of a compound according to claim 1, wherein said diseases are selected in the group including multiple sclerosis, psoriasis, rheumatoid arthritis, multiple sclerosis, systemic lupus erythematosis, inflammatory bowel disease, lung inflammation, thrombosis or brain infection/inflammation such as meningitis or encephalitis.

3. Use of a compound according to claim 1 wherein said diseases are selected in the group including Alzheimer's disease, Huntington's disease, CNS trauma, stroke or ischemic conditions.

4. Use of a compound according to claim 1, wherein said diseases are selected in the group including atherosclerosis, heart hypertrophy, cardiac myocyte dysfunction, elevated blood pressure or vasoconstriction.

5. Use of a compound according to claim 1, wherein said diseases are selected in the group including chronic obstructive pulmonary disease, anaphylactic shock fibrosis, psoriasis, allergic diseases, asthma, stroke or ischemic conditions, ischemia-reperfusion, platelets aggregation/activation, skeletal muscle atrophy/hypertrophy, leukocyte recruitment in cancer tissue, angiogenesis, invasion metastisis, in particular melanoma, Karposi's sarcoma, acute and chronic bacterial and viral infections, sepsis,, transplantation, graft rejection, glomerulo sclerosis, glomerulo nephritis, progressive renal fibrosis, endothelial and epithelial injuries in the lung or in general lung airways inflammation.

6. Use according to any of the precedent claims, wherein Y¹ and Y² are both oxygen.

7. Use according to any of the precedent claims, wherein n is 1 or 2 and R¹ and R² are both H.

8. Use of compounds according to any of the preceding claims, wherein X is S, Y¹ and Y² are both O, R¹ and R² are as above-defined and n is 0.

9. Use according to any of the precedent claims, whereby the thiazolidinone-vinyl fused-benzene derivative has the formula (Ia) 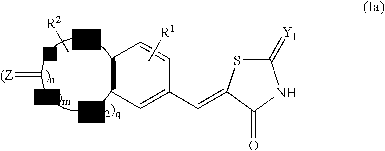
wherein Y¹, R¹, R², Z and n are as above defined;
V and W are each independently from each other O, S or —NR³ wherein R³ is H or C₁-C₆ alkyl;
G is a C₁-C₅ alkylene or a C₁-C₅ alkenylene group;
o and m are each independently from each other 0 or 1;
q is an integer from 0 to 4.

10. Use according to claim 9, whereby the thiazolidinone-vinyl fused-benzene derivative has the formula (Ib) 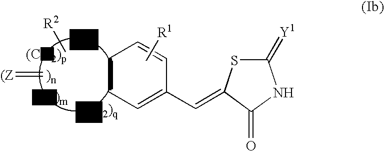
wherein Y¹, R¹, R², V, Z, W, m, n, o, q are as above defined and p is an integer from 1 to 4.

11. Use according to any of claims 9 or 10, whereby the thiazolidinone-vinyl fused-benzene derivative has the formula (Ic) 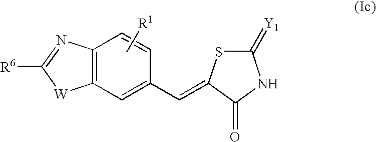
wherein W as well as R¹ and Y¹ are as above defined, R⁶ is H or OH.

12. Use according to any of claims 9 or 10, whereby the thiazolidinone-vinyl fused-benzene derivative has the formula (Id): 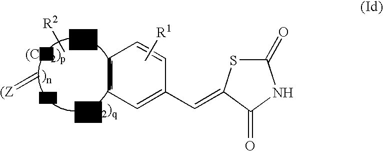
wherein R¹, R², Z and n are as above defined; m is 0 or 1;
p is an integer from 1 to 4 and q is an integer from 0 to 4.

13. Use of compounds according to any of claims 9, 10 or 12 wherein Z is O, m is 0, n is 1, p is 1 or 2, q is 1, R¹ and R² are each as above defined.

14. Use of compounds according to any of claims 9, 10 or 12 wherein m is 1, n is 0, p is 1 or 2, q is 0, R¹ and R² are each as above defined.

15. Use according to any of claims 9, 10 and 12 to 14 wherein m is 0, n is 1, p is 1 or 2, q is 0, R¹ and R² are each as defined in claim 1.

16. Use according to any of claims 9, 10 and 12 to 14 wherein R¹ is halogen or hydrogen.

17. Use according to any of claims 1 to 16 for the modulation, in particular for the inhibition, of the PI3 kinase activity.

18. Use according to claim 17, wherein said PI3 kinase is a PI3 kinase γ.

19. A thiazolidinone-vinyl fused-benzene derivative according to formula (II) 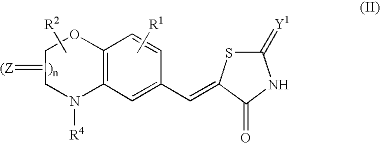
as well as its geometrical isomers, its optically active forms as enantiomers, diastereo-mers and its racemate forms, as well as pharmaceutically acceptable salts and pharma-ceutically active derivatives thereof, wherein
Z, Y¹, R¹, R² are as above defined, n is 0 or 1 and
R⁴ is selected in the group comprising or consisting of H, acyl, C₁-C₆-alkyl, C₂-C₆-alkenyl, C₂-C₆-alkynyl, C₁-C₆-alkyl carboxy, C₁-C₆-alkyl acyl, C₁-C₆-alkyl alkoxycarbonyl, C₁-C₆-alkyl aminocarbonyl, C₁-C₆-alkyl acyloxy, C₁-C₆-alkyl, acylamino, C₁-C₆-alkyl ureido, C₁-C₆-alkyl amino, C₁-C₆-alkyl alkoxy or C₁-C₆-alkyl sulfanyl, C₁-C₆-alkyl sulfinyl, C₁-C₆-alkyl sulfonyl, C₁-C₆-alkyl sulfonylaminoaryl aryl, heteroaryl, C₃-C₈-cycloalkyl or heterocycloalkyl, C₁-C₆-alkyl aryl, C₁-C₆-alkyl heteroaryl, C₂-C₆-alkenyl-aryl or -heteroaryl, C₂-C₆-alkynyl aryl or -heteroaryl, carboxy, hydroxy, C₁-C₆-alkoxy, C₁-C₆ alkyl carbamate, sulfonylamino, sulfanyl or sulfonyl.

20. A thiazolidinone-vinyl fused-benzene derivative according to claim 19, wherein Y¹ is O.

21. A thiazolidinone-vinyl fused-benzene derivative according to any claims 19 or 20, wherein R⁴ is selected in the group consisting of C₁-C₆-alkyl, C₁-C₆-alkyl aryl, C₁-C₆-alkyl heteroaryl, aryl, heteroaryl, C₃-C₈-cycloalkyl or heterocycloalkyl, C₁-C₆-alkyl aryl, C₁-C₆-alkyl heteroaryl, C₂-C₆-alkenyl-aryl or -heteroaryl or C₂-C₆-alkynyl aryl or -heteroaryl.

22. A thiazolidinone-vinyl fused-benzene derivative according to formula (III) 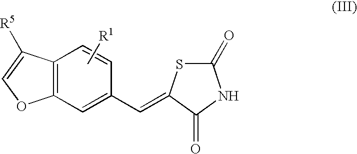
as well as its geometrical isomers, its optically active forms as enantiomers, diastereo-mers and its racemate forms, as well as pharmaceutically acceptable salts and pharma-ceutically active derivatives thereof,
wherein R¹ is as above defined and R⁵ is selected in the group comprising or consisting of H, halogen, acyl, amino, C₁-C₆-alkyl, C₂-C₆-alkenyl, C₂-C₆-alkynyl, C₁-C₆-alkyl carboxyl, C₁-C₆-alkyl acyl, C₁-C₆-alkyl alkoxycarbonyl, C₁-C₆-alkyl aminocarbonyl, C₁-C₆-alkyl acyloxy, C₁-C₆-alkyl, acylamino, C₁-C₆-alkyl ureido, C₁-C₆-alkyl amino, C₁-C₆-alkyl alkoxy or C₁-C₆-alkyl sulfanyl, C₁-C₆-alkyl sulfinyl, C₁-C₆-alkyl sulfonyl, C₁-C₆-alkyl sulfonylaminoaryl, aryl, heteroaryl, C₃-C₈-cycloalkyl or heterocycloalkyl, C₁-C₆-alkyl aryl, C₁-C₆-alkyl heteroaryl, C₂-C₆-alkenyl-aryl or -heteroaryl, C₂-C₆-alkynyl aryl or -heteroaryl, carboxy, cyano, hydroxy, C₁-C₆-alkoxy, nitro, acylamino, C₁-C₆ alkyl carbamate, ureido, sulfonylamino, sulfanyl or sulfonyl.

23. A thiazolidinone-vinyl fused-benzene derivative according to any of claims 19 to 22 for use as a medicament.

24. A pharmaceutical composition containing at least one thiazolidinone-vinyl fused-benzene derivative according to any of claims 19 to 22 and a pharmaceutically acceptable carrier, diluent or excipient thereof.

25. Use of a thiazolidinone-vinyl fused-benzene derivative according to any of claims 19 to 22 for the preparation of a medicament for the prophylaxis and/or treatment of autoimmune disorders and/or inflammatory diseases, cardiovascular diseases, neurodegenerative diseases, bacterial or viral infections, kidney diseases, platelet aggregation, cancer, transplantation, graft rejection or lung injuries.

26. Use of a thiazolidinone-vinyl fused-benzene derivative according to claim 25 wherein said diseases are selected in the group including multiple sclerosis, psoriasis, rheumatoid arthritis, multiple sclerosis, systemic lupus erythematosis, inflammatory bowel disease, lung inflammation, thrombosis or brain infection/inflammation such as meningitis or encephalitis.

27. Use of a thiazolidinone-vinyl fused-benzene derivative according to claim 25 wherein said diseases are selected in the group including Alzheimer's disease, Huntington's disease, CNS trauma, stroke or ischemic conditions.

28. Use of a thiazolidinone-vinyl fused-benzene derivative according to claim 25 wherein said diseases are selected in the group including atherosclerosis, heart hypertrophy, cardiac myocyte dysfunction, elevated blood pressure or vasoconstriction.

29. Use of a thiazolidinone-vinyl fused-benzene derivative according to claim 25 wherein said diseases are selected in the group including chronic obstructive pulmonary disease, anaphylactic shock fibrosis, psoriasis, allergic diseases, asthma, stroke or ischemic conditions, ischemia-reperfusion, platelets aggregation/activation, skeletal muscle atrophy/hypertrophy, leukocyte recruitment in cancer tissue, angiogenesis, invasion metastisis, in particular melanoma, Karposi's sarcoma, acute and chronic bacterial and viral infections, sepsis, , transplantation, graft rejection, glomerulo sclerosis, glomerulo nephritis, progressive renal fibrosis, endothelial and epithelial injuries in the lung or in general lung airways inflammation.

30. Use according to any of claims 25 to 29 for the modulation, particularly the inhibition of PI3Kinase activity.

31. Use according to claim 30 wherein said PI3Kinase is a PI3Kinase-γ.

32. A method of preparing a thiazolidinone-vinyl fused-benzene derivatives of formula (II) according to any of claims 19 to 21 comprising the following step: 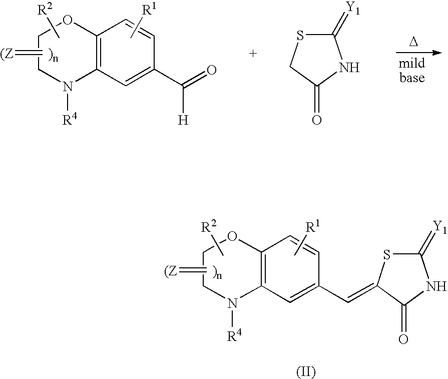
wherein R¹, R², R⁴, Y¹, Z and n are as above defined.

33. A method of preparing a thiazolidinone-vinyl fused-benzene derivatives of formula (III) according to claim 22 comprising the following step: 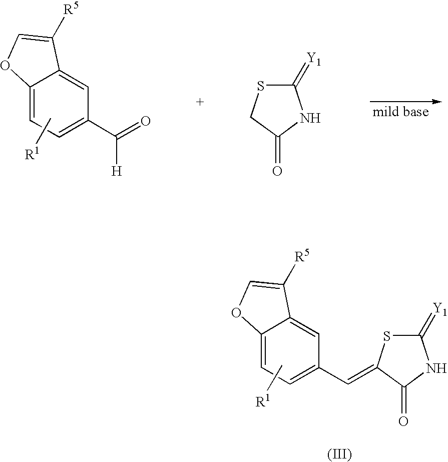
wherein R¹, R⁵and Y¹ are as above defined.
